(19) United States
(12) Patent Application Publication
(10) Pub. No.: US 2025/0134847 A1
(43) Pub. Date: May 1, 2025

(54) COMBINATION THERAPIES

(71) Applicant: QBIOTICS PTY LTD, Taringa, Queensland (AU)

(72) Inventors: Paul Warren REDDELL, Yungaburra, Queensland (AU); Peter Gordon PARSONS, St. Lucia, Queensland (AU); Glen Mathew BOYLE, Taringa, Queensland (AU); Jason Kingsley CULLEN, Alexandra Hills, Queensland (AU)

(21) Appl. No.: 18/838,666

(22) PCT Filed: Feb. 17, 2023
(86) PCT No.: PCT/AU2023/050108

§ 371 Date: Aug. 15, 2024

(30) Foreign Application Priority Data
Feb. 17, 2022 (AU) 2022900340

(51) Int. Cl. A61K 31/336; A61K 31/513; A61K 31/704; A61K 33/243; A61P 35/00

(52) U.S. Cl. CPC A61K 31/336; A61K 31/513; A61K 31/704; A61K 33/243; A61P 35/00

(57) ABSTRACT

Provided herein are combination therapies comprising an epoxytigliane compound (for example, tigilanol tiglate) and either irradiation or a chemotherapeutic agent that is i) a chemotherapeutic agent that damages DNA (for example, cisplatin), or ii) a chemotherapeutic agent that inhibits tumour-associated host-derived cells that support the growth and/or invasion of tumour cells (for example, 5-fluoruracil, or doxorubicin). Pharmaceutical compositions and kits containing epoxytigliane compounds and the chemotherapeutic agents are also provided.

The present application claims priority from Australian Provisional Patent Application No. 2022900340 filed 17 Feb. 2022, the entirety of which is incorporated herein by reference.

### FIELD

The present invention relates to combination therapies comprising an epoxytigliane compound and a cancer chemotherapeutic agent or irradiation. Pharmaceutical compositions and kits containing epoxytigliane compounds and the cancer chemotherapeutic agents are also described.

### BACKGROUND

The treatment of cancer has many options including chemotherapeutic agents, irradiation, surgery and immune stimulating agents. Some therapies are developed for particular types of cancer or those defined by particular biological markers. Known therapies often have significant side effects and may not be suitable for some patients. For example, some tumours are inoperable and some patients have co-morbidities that reduce the effectiveness of a chemotherapeutic agent or prevent the use of particular therapeutic agents because of side effects.

One option for reducing side effects, expanding the therapies available to patients with co-morbidities and reducing the development of resistance to a particular therapy is to use combinations of therapeutic agents. The effects of a combination of therapeutic agents may be additive, synergistic or antagonistic and it is not always predictable whether a combination is likely to be additive, synergistic or antagonistic, or whether reducing therapeutic dosages will be effective.

There is a need for combination therapies that provide patients with benefits such as lower dosages, reduced side effects, synergistic effects, increased therapy options and/or improved treatment success rates.

Epoxytiglienone compounds have potent anti-tumour activity. When administered intra-tumourally, epoxytiglienones initiate rapid haemorrhagic necrosis of the tumour mass by directly disrupting tumour vasculature (Boyle et al. 2014).

The present disclosure is predicated at least in part on the discovery that the epoxytigliane compound, Compound 1, is able to synergise with cisplatin, 5 fluorouracil, doxorubicin and irradiation in a variety of cancers, including squamous cell carcinomas, adenocarcinomas and fibrosarcomas.

### SUMMARY

In a first aspect, there is provided a method of treating a tumour comprising administering a combination of an epoxytigliane compound and a second therapeutic agent; wherein the epoxytigliane compound is a compound of formula (I):

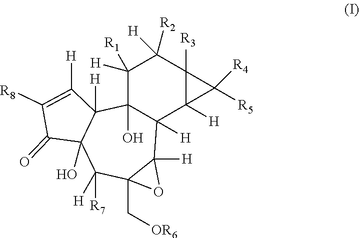

or a geometric isomer or stereoisomer or a pharmaceutically acceptable salt thereof, wherein
R₁ is hydrogen or C₁₋₆alkyl;
R₂ is —OH or —OR₉;
R₃ is —OH or —OR₉;
R₄ and R₅ are independently selected from hydrogen and C₁₋₆alkyl;
R₆ is hydrogen or —R₁₀;
R₇ is hydroxy or OR₁₀;
R₈ is hydrogen or C₁₋₆alkyl;
R₉ is —C₁₋₂₀alkyl, —C₂₋₂₀alkenyl, —C₂₋₂₀alkynyl, —C(O)C₁₋₂₀alkyl, —C(O)C₂₋₂₀alkenyl, —C(O)C₂₋₂₀alkynyl, —C(O)cycloalkyl, —C(O)C₁₋₁₀alkylcycloalkyl; —C(O)C₂₋₁₀alkenylcycloalkyl, —C(O)₂₋₁₀alkynylcycloalkyl, —C(O)aryl, —C(O)C₁₋₁₀alkylaryl, —C(O)C₂₋₁₀alkenylaryl, —C(O)C₂₋₁₀alkynylaryl, —C(O)C₁₋₁₀alkylC(O)R₁₁, —C(O)C₂₋₁₀alkenylC(O)R₁₁, —C(O)C₂₋₁₀alkynylC(O)R₁₁, —C(O)C₁₋₁₀alkylCH(OR₁₁)(OR₁₁), —C(O)C₂₋₁₀alkenylCH(OR₁₁)(OR₁₁), —C(O)C₂₋₁₀alkynylCH(OR₁₁)(OR₁₁), —C(O)C₁₋₁₀alkylSR₁₁, —C(O)C₂₋₁₀alkenylSR₁₁, —C(O)C₂₋₁₀alkynylSR₁₁, —C(O)C₁₋₁₀alkylC(O)OR₁₁, —C(O)C₂₋₁₀alkenylC(O)OR₁₁, —C(O)C₂₋₁₀alkynylC(O)OR₁₁, —C(O)C₁₋₁₀alkylC(O)SR₁₁, —C(O)C₂₋₁₀alkenylC(O)SR₁₁, —C(O)C₂₋₁₀alkynylC(O)SR₁₁,

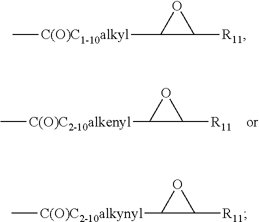

R₁₀ is —C₁₋₆alkyl, —C₂₋₆alkenyl, —C₂₋₆alkynyl, —C(O)C₁₋₆alkyl, —C(O)C₂₋₆alkenyl, —C(O)C₂₋₆alkynyl, —C(O)aryl, —C(O)C₁₋₆alkylaryl, —C(O)C₂₋₆alkenylaryl, or —C(O)C₂₋₆alkynylaryl; and
R₁₁ is hydrogen, —C₁₋₁₀alkyl, —C₂₋₁₀alkenyl, —C₂₋₁₀alkynyl, cycloalkyl or aryl;
wherein each alkyl, alkenyl, alkynyl, cycloalkyl or aryl group is optionally substituted;
   and
   wherein the second therapeutic agent is selected from:
i) a chemotherapeutic agent that damages DNA;
ii) a chemotherapeutic agent that inhibits tumour-associated host-derived cells that support the growth and/or invasion of tumour cells; and
iii) irradiation.

In another aspect, there is provided a combination of epoxytigliane compound and second therapeutic agent for use in the treatment of a tumour, wherein the epoxytigliane compound is a compound of formula (I):

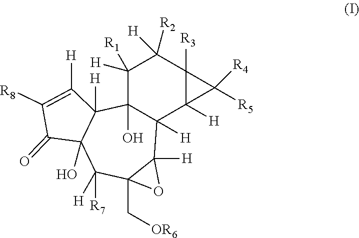

or a geometric isomer or stereoisomer or a pharmaceutically acceptable salt thereof,
wherein
R₁ is hydrogen or C₁₋₆alkyl;
R₂ is —OH or —OR₉;
R₃ is —OH or —OR₉;
R₄ and R₅ are independently selected from hydrogen and C₁₋₆alkyl;
R₆ is hydrogen or —R₁₀;
R₇ is hydroxy or OR₁₀;
R₈ is hydrogen or C₁₋₆alkyl;
R₉ is —C₁₋₂₀alkyl, —C₂₋₂₀alkenyl, —C₂₋₂₀alkynyl, —C(O)C₁₋₂₀alkyl, —C(O)C₂₋₂₀alkenyl, —C(O)C₂₋₂₀alkynyl, —C(O)cycloalkyl, —C(O)C₁₋₁₀alkylcycloalkyl; —C(O)C₂₋₁₀alkenylcycloalkyl, —C(O)C₂₋₁₀alkynylcycloalkyl, —C(O)aryl, —C(O)C₁₋₁₀alkylaryl, —C(O)C₂₋₁₀alkenylaryl, —C(O)C₂₋₁₀alkynylaryl, —C(O)C₁₋₁₀alkylC(O)R₁₁, —C(O)C₂₋₁₀alkenylC(O)R₁₁, —C(O)C₂₋₁₀alkynylC(O)R₁₁, —C(O)C₁₋₁₀alkylCH(OR₁₁)(OR₁₁), —C(O)C₂₋₁₀alkenylCH(OR₁₁)(OR₁₁), —C(O)C₂₋₁₀alkynylCH(OR₁₁)(OR₁₁), —C(O)C₁₋₁₀alkylSR₁₁, —C(O)C₂₋₁₀alkenylSR₁₁, —C(O)C₂₋₁₀alkynylSR₁₁, —C(O)C₁₋₁₀alkylC(O)OR₁₁, —C(O)C₂₋₁₀alkenylC(O)OR₁₁, —C(O)C₂₋₁₀alkynylC(O)OR₁₁, —C(O)C₁₋₁₀alkylC(O)SR₁₁, —C(O)C₂₋₁₀alkenylC(O)SR₁₁, —C(O)C₂₋₁₀alkynylC(O)SR₁₁,

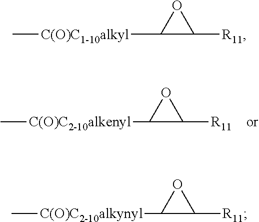

R₁₀ is —C₁₋₆alkyl, —C₂₋₆alkenyl, —C₂₋₆alkynyl, —C(O)C₁₋₆alkyl, —C(O)C₂₋₆alkenyl, —C(O)C₂₋₆alkynyl, —C(O)aryl, —C(O)C₁₋₆alkylaryl, —C(O)C₂₋₆alkenylaryl, or —C(O)C₂₋₆alkynylaryl; and
R₁₁ is hydrogen, —C₁₋₁₀alkyl, —C₂₋₁₀alkenyl, —C₂₋₁₀alkynyl, cycloalkyl or aryl;
wherein each alkyl, alkenyl, alkynyl, cycloalkyl or aryl group is optionally substituted;
   and
   wherein the second therapeutic agent is selected from:
i) a chemotherapeutic agent that damages DNA;
ii) a chemotherapeutic agent that inhibits tumour-associated host-derived cells that support the growth and/or invasion of tumour cells; and
iii) irradiation.

In a further aspect, there is provided a use of an epoxytigliane compound in the manufacture of a first medicament and a second chemotherapeutic agent in the manufacture of a second medicament as a combination therapy for treating a tumour; wherein the epoxytigliane compound is a compound of formula (I):

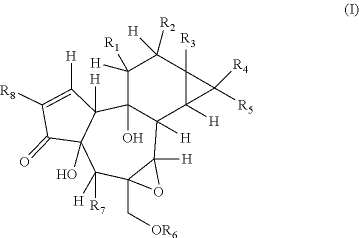

or a geometric isomer or stereoisomer or a pharmaceutically acceptable salt thereof;
wherein
R₁ is hydrogen or C₁₋₆alkyl;
R₂ is —OH or —OR₉;
R₃ is —OH or —OR₉;
R₄ and R₅ are independently selected from hydrogen and C₁₋₆alkyl;
R₆ is hydrogen or —R₁₀;
R₇ is hydroxy or OR₁₀;
R₈ is hydrogen or C₁₋₆alkyl;
R₉ is —C₁₋₂₀alkyl, —C₂₋₂₀alkenyl, —C₂₋₂₀alkynyl, —C(O)C₁₋₂₀alkyl, —C(O)C₂₋₂₀alkenyl, —C(O)C₂₋₂₀alkynyl, —C(O)cycloalkyl, —C(O)C₁₋₁₀alkylcycloalkyl; —C(O)C₂₋₁₀alkenylcycloalkyl, —C(O)C₂₋₁₀alkynylcycloalkyl, —C(O)aryl, —C(O)C₁₋₁₀alkylaryl, —C(O)C₂₋₁₀alkenylaryl, —C(O)C₂₋₁₀alkynylaryl, —C(O)C₁₋₁₀alkylC(O)R₁₁, —C(O)C₂₋₁₀alkenylC(O)R₁₁, —C(O)C₂₋₁₀alkynylC(O)R₁₁, —C(O)C₁₋₁₀alkylCH(OR₁₁)(OR₁₁), —C(O)C₂₋₁₀alkenylCH(OR₁₁)(OR₁₁), —C(O)C₂₋₁₀alkynylCH(OR₁₁)(OR₁₁), —C(O)C₁₋₁₀alkylSR₁₁, —C(O)C₂₋₁₀alkenylSR₁₁, —C(O)C₂₋₁₀alkynylSR₁₁, —C(O)C₁₋₁₀alkylC(O)OR₁₁, —C(O)C₂₋₁₀alkenylC(O)OR₁₁, —C(O)C₂₋₁₀alkynylC(O)OR₁₁, —C(O)C₁₋₁₀alkylC(O)SR₁₁, —C(O)C₂₋₁₀alkenylC(O)SR₁₁, —C(O)C₂₋₁₀alkynylC(O)SR₁₁,

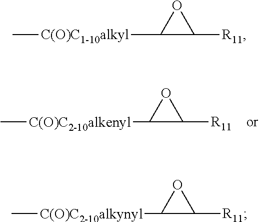

R₁₀ is —C₁₋₆alkyl, —C₂₋₆alkenyl, —C₂₋₆alkynyl, —C(O)C₁₋₆alkyl, —C(O)C₂₋₆alkenyl, —C(O)C₂₋₆alkynyl, —C(O)aryl, —C(O)C₁₋₆alkylaryl, —C(O)C₂₋₆alkenylaryl, or —C(O)C₂₋₆alkynylaryl; and
R₁₁ is hydrogen, —C₁₋₁₀alkyl, —C₂₋₁₀alkenyl, —C₂₋₁₀alkynyl, cycloalkyl or aryl; wherein each alkyl, alkenyl, alkynyl, cycloalkyl or aryl group is optionally substituted;
   and
   wherein the second chemotherapeutic agent is selected from:
i) a chemotherapeutic agent that damages DNA; and
ii) a chemotherapeutic agent that inhibits tumour-associated host-derived cells that support the growth and/or invasion of tumour cells.

In yet another aspect, there is provided a use of an epoxytigliane compound in the manufacture of a medicament in a combination therapy with irradiation for treating a tumour; wherein the epoxytigliane compound is a compound of formula (I):

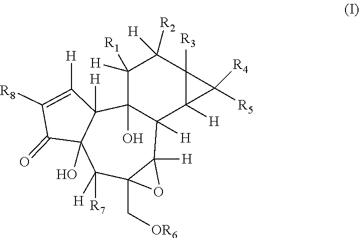

or a geometric isomer or stereoisomer or a pharmaceutically acceptable salt thereof, wherein
R₁ is hydrogen or C₁₋₆alkyl;
R₂ is —OH or —OR₉;
R₃ is —OH or —OR₉;
R₄ and R₅ are independently selected from hydrogen and C₁₋₆alkyl;
R₆ is hydrogen or —R₁₀;
R₇ is hydroxy or OR₁₀;
R₈ is hydrogen or C₁₋₆alkyl;
R₉ is —C₁₋₂₀alkyl, —C₂₋₂₀alkenyl, —C₂₋₂₀alkynyl, —C(O)C₁₋₂₀alkyl, —C(O)C₂₋₂₀alkenyl, —C(O)C₂₋₂₀alkynyl, —C(O)cycloalkyl, —C(O)C₁₋₁₀alkylcycloalkyl; —C(O)C₂₋₁₀alkenylcycloalkyl, —C(O)C₂₋₁₀alkynylcycloalkyl, —C(O)aryl, —C(O)C₁₋₁₀alkylaryl, —C(O)C₂₋₁₀alkenylaryl, —C(O)C₂₋₁₀alkynylaryl, —C(O)C₁₋₁₀alkylC(O)R₁₁, —C(O)C₂₋₁₀alkenylC(O)R₁₁, —C(O)C₂₋₁₀alkynylC(O)R₁₁, —C(O)C₁₋₁₀alkylCH(OR₁₁)(OR₁₁), —C(O)C₂₋₁₀alkenylCH(OR₁₁)(OR₁₁), —C(O)C₂₋₁₀alkynylCH(OR₁₁)(OR₁₁), —C(O)C₁₋₁₀alkylSR₁₁, —C(O)C₂₋₁₀alkenylSR₁, —C(O)C₂₋₁₀alkynylSR₁₁, —C(O)C₁₋₁₀alkylC(O)OR₁₁, —C(O)C₂₋₁₀alkenylC(O)OR₁₁, —C(O)C₂₋₁₀alkynylC(O)OR₁₁, —C(O)C₁₋₁₀alkylC(O)SR₁₁, —C(O)C₂₋₁₀alkenylC(O)SR₁₁, —C(O)C₂₋₁₀alkynylC(O)SR₁₁,

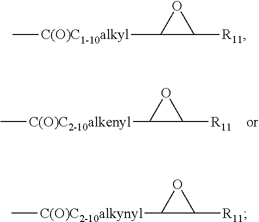

R₁₀ is —C₁₋₆alkyl, —C₂₋₆alkenyl, —C₂₋₆alkynyl, —C(O)C₁₋₆alkyl, —C(O)C₂₋₆alkenyl, —C(O)C₂₋₆alkynyl, —C(O)aryl, —C(O)C₁₋₆alkylaryl, —C(O)C₂₋₆alkenylaryl, or —C(O)C₂₋₆alkynylaryl; and
R₁₁ is hydrogen, —C₁₋₁₀alkyl, —C₂₋₁₀alkenyl, —C₂₋₁₀alkynyl, cycloalkyl or aryl;
wherein each alkyl, alkenyl, alkynyl, cycloalkyl or aryl group is optionally substituted.

### BRIEF DESCRIPTION OF FIGURES

FIG. 1a is a plot of individual SCC-15 tumour volumes following treatment with combinations of intratumoural Compound 1, intraperitoneal cisplatin and control agents in the BALB/c Foxn1^{nu} xenogeneic mouse model over time.

FIG. 1b is a plot of average SCC-15 tumour volumes following treatment with combinations of intratumoural Compound 1, intraperitoneal cisplatin and control agents in the BALB/c Foxn1^{nu} xenogeneic mouse model over time.

FIG. 2 is a plot of Kaplan-Meier survival curves for SCC-15 tumours following treatment with combinations of intratumoural Compound 1, intraperitoneal cisplatin and control agents in the BALB/c Foxn1^{nu} xenogeneic mouse model over time.

FIG. 3a is a plot of individual UV-13-1 tumour volumes following treatment with combinations of intratumoural Compound 1, intraperitoneal cisplatin and control agents in the C3H/HeNCr syngeneic mouse model over time.

FIG. 3b is a plot of average UV-13-1 tumour volumes following treatment with combinations of intratumoural Compound 1, intraperitoneal cisplatin and control agents in the C3H/HeNCr syngeneic mouse model over time.

FIG. 4 is a plot of Kaplan-Meier survival curves for UV-13-1 tumours following treatment with combinations of intratumoural Compound 1, intraperitoneal cisplatin and control agents in the C3H/HeNCr syngeneic mouse model over time.

FIG. 5a is a plot of individual SCC-15 tumour volumes following treatment with combinations of intratumoural Compound 1, external beam irradiation and control agents in the BALB/c Foxn1^{nu} xenogeneic mouse model over time.

FIG. 5b is a plot of average SCC-15 tumour volumes following treatment with combinations of intratumoural Compound 1, external beam irradiation and control agents in the BALB/c Foxn1^{nu} xenogeneic mouse model over time.

FIG. 6 is a plot of Kaplan-Meier survival curves for SCC-15 tumours following treatment with combinations of intratumoural Compound 1, external beam irradiation and control agents in the BALB/c Foxn1^{nu} xenogeneic mouse model over time.

FIG. 7a is a plot of individual MC38 tumour volumes following treatment with combinations of intratumoural Compound 1, external beam irradiation and control agents in the C57BL/6 syngeneic mouse model over time.

FIG. 7b is a plot of average MC38 tumour volumes following treatment with combinations of intratumoural Compound 1, external beam irradiation and control agents in the C57BL/6 syngeneic mouse model over time.

FIG. 8 is a plot of Kaplan-Meier survival curves for MC38 tumours following treatment with combinations of intratumoural Compound 1, intraperitoneal cisplatin and control agents in the C57BL/6 syngeneic mouse model over time.

FIG. 9a is a plot of cell survival for A431 cells following concurrent treatment with combinations of cisplatin and different concentrations of Compound 1 with each data point representing the mean of three different experiments.

FIG. 9b is a plot of cell survival for SCC-15 cells following concurrent treatment with combinations of cisplatin and different concentrations of Compound 1 with each data point representing the mean of three different experiments.

FIG. 10a is a plot of cell survival for A431 cells following concurrent treatment with combinations of 5-fluorouracil and different concentrations of Compound 1 with each data point representing the mean of three different experiments.

FIG. 10b is a plot of cell survival for SCC-15 cells following concurrent treatment with combinations of 5-fluorouracil and different concentrations of Compound 1 with each data point representing the mean of three different experiments.

FIG. 11a is a plot of cell survival for A431 cells following concurrent treatment with combinations of Compound 1 and different concentrations of irradiation with each data point representing the mean of three different experiments.

FIG. 11b is a plot of cell survival for SCC-15 cells following concurrent treatment with combinations of Compound 1 and different concentrations of irradiation with each data point representing the mean of three different experiments.

FIG. 12 is a Combination-Effect (Fa-CI) plot for A431 cells demonstrating a relationship between Compound 1 and cisplatin, 5-fluorouracil and irradiation.

FIG. 13 is a Combination-Effect (Fa-CI) plot for SCC-15 cells demonstrating relationship between Compound 1 and cisplatin, 5-fluorouracil and irradiation.

FIG. 14a is a plot of Kaplan-Meier survival curves for B16-F10-OVA tumours having a tumour size greater than 100 mm³ following treatment with combinations of intratumoural Compound 1 (5 μg or 10 μg) with intraperitoneal doxorubicin, and control agents in C57BL/6 mice overtime.

FIG. 14b is a plot of Kaplan-Meier survival curves for B16-F10-OVA tumours (overall survival) following treatment with combinations of intratumoural Compound 1 (5 μg or 10 μg) with intraperitoneal doxorubicin, and control agents in C57BL/6 mice over time.

FIG. 15a is a plot of Kaplan-Meier survival curves for Lewis Lung Carcinoma (LLC) tumours having a tumour size greater than 100 mm³ following treatment with combinations of intratumoural Compound 1 (5 μg or 10 μg) with intraperitoneal doxorubicin, and control agents in C57BL/6 mice over time.

FIG. 15b is a plot of Kaplan-Meier survival curves for Lewis Lung Carcinoma (LLC) tumours (overall survival) following treatment with combinations of intratumoural Compound 1 (5 μg or 10 μg) with intraperitoneal doxorubicin, and control agents in C57BL/6 mice over time.

### DETAILED DESCRIPTION

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The present disclosure refers to the entire contents of certain documents being incorporated herein by reference. In the event of any inconsistent teaching between the teaching of the present disclosure and the contents of those documents, the teaching of the present disclosure takes precedence.

It is to be understood that if any prior art publication is referred to herein, such reference does not constitute an admission that the publication forms a part of the common general knowledge in the art in Australia or any other country.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 25%, 20%, 15% or 10% to a reference quantity, level, value, dimension, size, or amount.

As used herein, the term “and/or”, e.g., “X and/or Y” shall be understood to mean either “X and Y” or “X or Y” and shall be taken to provide explicit support for both meanings or for either meaning.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

Unless otherwise indicated, terms such as “first,” “second,” etc. are used herein merely as labels, and are not intended to impose ordinal, positional, or hierarchical requirements on the items to which these terms refer. Moreover, reference to a “second” item does not require or preclude the existence of lower-numbered item (e.g., a “first” item) and/or a higher-numbered item (e.g., a “third” item).

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example and without limitation, two of item A, one of item B, and ten of item C; four of item B and seven of item C; or some other suitable combination.

The term “alkyl” refers to optionally substituted linear and branched hydrocarbon groups having 1 to 20 carbon atoms. Where appropriate, the alkyl group may have a specified number of carbon atoms, for example, —C₁-C₆ alkyl which includes alkyl groups having 1, 2, 3, 4, 5 or 6 carbon atoms in linear or branched arrangements. Non-limiting examples of alkyl groups include methyl, ethyl, propyl, isopropyl, butyl, s- and t-butyl, pentyl, 2-methylbutyl, 3-methylbutyl, hexyl, 2-methylpentyl, 3-methylpentyl, 4-methylpentyl, 2-ethylbutyl, 3-ethylbutyl, heptyl, octyl, nonyl, decyl, undecyl, dodecyl, tridecyl, tetradecyl and pentadecyl.

The term “alkenyl” refers to optionally substituted, unsaturated linear or branched hydrocarbons, having 2 to 20 carbon atoms and having at least one double bond. Where appropriate, the alkenyl group may have a specified number of carbon atoms, for example, C₂-C₆ alkenyl which includes alkenyl groups having 2, 3, 4, 5 or 6 carbon atoms in linear or branched arrangements. Non-limiting examples of alkenyl groups include, ethenyl, propenyl, isopropenyl, butenyl, s- and t-butenyl, pentenyl, hexenyl, hept-1,3-diene, hex-1,3-diene, non-1,3,5-triene and the like.

The term “alkynyl” refers to optionally substituted unsaturated linear or branched hydrocarbons, having 2 to 20 carbon atoms, having at least one triple bond. Where appropriate, the alkynyl group may have a specified number of carbon atoms, for example, C₂-C₆ alkynyl which includes alkynyl groups having 2, 3, 4, 5 or 6 carbon atoms in linear or branched arrangements. Non-limiting examples include ethynyl, propynyl, butynyl, pentynyl and hexynyl.

The terms “cycloalkyl” and “carbocyclic” refer to optionally substituted saturated or unsaturated mono-cyclic, bicyclic or tricyclic hydrocarbon groups. Where appropriate, the cycloalkyl group may have a specified number of carbon atoms, for example, C₃-C₆ cycloalkyl is a carbocyclic group having 3, 4, 5 or 6 carbon atoms. Non-limiting examples may include cyclopropyl, cyclobutyl, cyclopentyl, cyclopentenyl, cyclohexyl, cyclohexenyl, cyclohexadienyl and the like.

“Aryl” means a C₆-C₁₄ membered monocyclic, bicyclic or tricyclic carbocyclic ring system having up to 7 atoms in each ring, wherein at least one ring is aromatic. Examples of aryl groups include, but are not limited to, phenyl, naphthyl, tetrahydronaphthyl, indanyl and biphenyl. The aryl may comprise 1-3 benzene rings. If two or more aromatic rings are present, then the rings may be fused together, so that adjacent rings share a common bond.

Each alkyl, alkenyl, alkynyl, cycloalkyl or aryl whether an individual entity or as part of a larger entity may be optionally substituted with one or more optional substituents selected from the group consisting of C₁₋₆alkyl, C₂₋₆alkenyl, C₃₋₆cycloalkyl, oxo (═O), —OH, —SH, C₁₋₆alkylO—, C₂₋₆alkenylO—, C₃₋₆cycloalkylO—, C₁₋₆alkylS—, C₂₋₆alkenylS—, C₃₋₆cycloalkylS—, —CO₂H, —CO₂C₁₋₆alkyl, —NH₂, —NH(C₁₋₆alkyl), —N(C₁₋₆alkyl)₂, —NH(phenyl), —N(phenyl)₂, —CN, —NO₂, -halogen, —CF₃, —OCF₃, —SCF₃, —CHF₂, —OCHF₂, —SCHF₂, -phenyl, —C₁₋₆ alkylphenyl, —Ophenyl, —C(O)phenyl, —C(O)C₁₋₆alkyl. Examples of suitable substituents include, but are not limited to, methyl, ethyl, propyl, isopropyl, butyl, sec-butyl, tert-butyl, vinyl, methoxy, ethoxy, propoxy, isopropoxy, butoxy, methylthio, ethylthio, propylthio, isopropylthio, butylthio, hydroxy, hydroxymethyl, hydroxyethyl, hydroxypropyl, hydroxybutyl, fluoro, chloro, bromo, iodo, cyano, nitro, —CO₂H, —CO₂CH₃, —C(O)CH₃, trifluoromethyl, trifluoromethoxy, trifluoromethylthio, difluoromethyl, difluoromethoxy, difluoromethylthio, morpholino, amino, methylamino, dimethylamino, phenyl, phenoxy, phenylcarbonyl, benzyl and acetyl.

The epoxytigliane compounds may be in the form of pharmaceutically acceptable salts. It will be appreciated however that non-pharmaceutically acceptable salts also fall within the scope of the invention since these may be useful as intermediates in the preparation of pharmaceutically acceptable salts or may be useful during storage or transport. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

It will also be recognised that the epoxytigliane compounds may possess asymmetric centres and are therefore capable of existing in more than one stereoisomeric form. The invention thus also relates to compounds in substantially pure isomeric form at one or more asymmetric centres e.g., greater than about 90% ee, such as about 95% or 97% ee or greater than 99% ee, as well as mixtures, including racemic mixtures, thereof. Such isomers may be obtained by isolation from natural sources, by asymmetric synthesis, for example using chiral intermediates, or by chiral resolution. The compounds of the invention may exist as geometrical isomers. The invention also relates to compounds in substantially pure cis (Z) or trans (E) forms or mixtures thereof.

The compounds of the present invention may be obtained by isolation from a plant or plant part, or by derivatisation of the isolated compound, or by derivatisation of a related compound. Isolation procedures and derivatisation procedures may be found in WO 2007/070985 and WO2014/169356.

The term “epoxytigliane compound” refers to a generally to a compound having the following basic carbon cyclic structure:

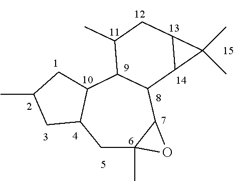

The compounds have a tricyclo[9.3.0.0]tetradecane system with a fused cyclopropane ring appended to the six membered ring. The epoxide is fused to the seven membered ring in the 6,7-position. The epoxytigliane compound is generally an epoxytiglien-3-one compound. The term “epoxytiglien-3-one compound” refers to a compound having an epoxy-tigliane structure defined above where the five membered ring has a 1,2-ene-3-one structure:

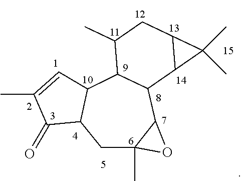

Throughout the specification, the term “epoxytigliane compound” is used to refer to epoxytiglienone compounds of formula (I).

The term “in combination with” as used herein refers to the epoxytigliane compound and the second chemotherapeutic agent (e.g. cancer chemotherapeutic agent) being administered in a single composition, or the second chemotherapeutic agent (e.g. cancer chemotherapeutic agent) or irradiation is administered separately, either simultaneously or sequentially. The epoxytigliane compound and the second chemotherapeutic agent (e.g. cancer chemotherapeutic agent) or irradiation may be administered at different times and different frequencies but in combination they exert biological effects at the same time or at overlapping times.

### Methods of Treatment

The present invention relates to methods of treating tumours, the method comprising the administration of an epoxytigliane compound or a pharmaceutically acceptable salt thereof in combination with second chemotherapeutic agent or irradiation.

In some embodiments, the tumour being treated is a tumour to which the epoxytigliane compound may be delivered in a localised way directly to the tumour. In particular embodiments, the tumour is a cutaneous tumour or subcutaneous tumour or a tumour accessible from the outside of the body, for example, a tumour that is palpable. In other embodiments, the tumour is an internal tumour. In some embodiments where the tumour is an internally located tumour, the localised delivery is achieved during surgery when the tumour is exposed and able to be injected with the epoxytigliane compound. In other embodiments, the tumour is internally located and the epoxytigliane compound is delivered by injection guided by an imaging technique, for example, guided by endoscopic ultrasound or by stereotactic imaging.

In some embodiments, the epoxytigliane compound is delivered to one or more tumours systemically.

In some embodiments, the tumour is a benign tumour. In other embodiments, the tumour is a malignant tumour. In some embodiments, the tumour is a primary tumour and in other embodiments, the tumour is a metastatic tumour. In some embodiments the tumour is a cutaneous tumour. Examples of cutaneous tumours include seborrheic keratosis, actinic keratosis, basal cell carcinoma (BCC) including nodular BCC, superficial BCC, infiltrative BCC and micronodular BCC, squamous cell carcinoma (SCC), including in-situ SCC and invasive SCC, adenocarcinoma, melanoma including superficial spreading melanoma, nodular melanoma, lentigo maligna melanoma, acral lentginous melanoma and desmoplastic/neutropic melanoma, sarcomas, cutaneous B cell lymphoma and cutaneous T cell lymphoma. Examples of cutaneous and subcutaneous tumours that may be treated include angiokeratoma, pyogenic granuloma, cherry angioma, glomus tumour, angiosarcoma, karposi sarcoma, Ewings sarcoma, malignant fibrous histiocytoma, leiomyosarcoma, rhabdomyosarcoma, liposarcoma, synovial sarcoma, stromal sarcoma, gastrointestinal stromal sarcoma, malignant peripheral nerve sheath tumour, primitive neuroectodermal tumour, neurofibroma, Merkel cell carcinoma, dermatofibroma, fibrosarcoma, epithelioid sarcoma and mastocytoma (mast cell tumour).

Where the tumour is an internal tumour, the internal tumour may be any tumour that is accessible to injection during surgery or by guided injection or one that may be treated with systemically administered epoxytigliane and include tumours of the brain, lung, colon, epidermoid, squamous cell, bladder, stomach, pancreas, breast, head, neck, renal system, kidney, liver, ovary, prostate, uterus, oesophagus, testicles, cervix, vagina, thyroid or skin.

In some embodiments, the tumour is a tumour of the head or neck. In some embodiments, the head or neck tumour is a cutaneous tumour. In other embodiments, the head or neck tumour is a mucosal epithelial tumour, for example, occurring in the mucosal epithelium of the upper aerodigestive tract. In particular embodiments, the tumour is a head and neck squamous cell carcinoma (HNSCC).

In some embodiments, the tumour is a mucosal head or neck cancer, especially a mucosal squamous cell carcinoma (SCC). In some embodiments, the mucosal tumour is associated with smoking and/or alcohol use. In some embodiments, the tumour is of the oropharynx, especially the tonsils or tongue base.

In some embodiments the tumour is associated with human papillomavirus (HPV). In particular embodiments, the HPV associated tumour is a mucosal SCC. HPV associated mucosal SCCs include tumours of the oropharynx, vulva, vagina and cervix.

In some embodiments, the tumour is a non-melantotic skin cancer (NMSC). The NMSC may occur on any part of the body. The tumour may be associated with ultraviolet solar radiation exposure, skin phenotype, immunosuppression, xeroderma pigmentosum, chemical exposure, chronic ulcers and scars, ionizing radiation or a combination thereof. In some embodiments, the tumour is a high risk tumour having one or more of the following: a size of greater than 4 cm, a depth >2 mm, presence of perineural or lymphovascular invasion, affecting ears or lips, patient immunosuppression and previous radiotherapy.

In some embodiments, the tumour is a squamous cell tumour (SCC), for example, a thyroid carcinoma, esophageal carcinoma, SCC of the lung, SCC of the penis (that may be associated with HPV) including Bowen's disease, Erythroplasia of Queyrat and Bowenoid papulosis, prostate cancer, vaginal cancer, cervical cancer and bladder cancer, particularly bladder cancer associated with schistosomiasis.

In some embodiments, the tumour is a sarcoma, for example, an angiosarcoma, karposi sarcoma, Ewings sarcoma, leiomyosarcoma, rhabdomyosarcoma, liposarcoma, synovial sarcoma, stromal sarcoma, gastrointestinal stromal sarcoma, fibrosarcoma or an epithelioid sarcoma. In some embodiments, the sarcoma is a UV induced fibrosarcoma.

In other embodiments, the tumour is an adenocarcinoma. Examples of adenocarcinoma include esophageal cancers, pancreatic cancer, prostate cancer, cervical cancer, stomach cancer, invasive ductal breast carcinoma, ductal breast carcinoma in situ, invasive lobular breast carcinoma, colon cancer, colorectal cancer, adenocarcinoma of the lung, cholangiocarcinoma (bile duct cancer) and vaginal cancer.

### Epoxytigliane Compounds

The epoxytigliane compound is a 6,7-epoxytigli-1,2-en-3-one compound of formula (I):

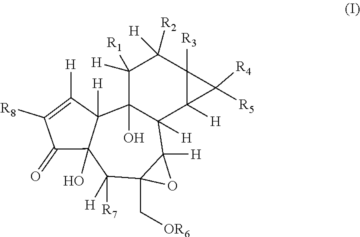

or a geometric isomer or stereoisomer or a pharmaceutically acceptable salt thereof,
wherein
R₁ is hydrogen or C₁₋₆alkyl;
R₂ is —OH or —OR₉;
R₃ is —OH or —OR₉;
R₄ and R₅ are independently selected from hydrogen and C₁₋₆alkyl;
R₆H is hydrogen or —R₁₀;
R₇ is hydroxy or —OR₁₀;
R₈ is hydrogen or C₁₋₆alkyl;
R₉ is —C₁₋₂₀alkyl, —C₂₋₂₀alkenyl, —C₂₋₂₀alkynyl, —C(O)C₂₋₂₀alkyl, —C(O)C₂₋₂₀alkenyl, —C(O)C₂₋₂₀alkynyl, —C(O)cycloalkyl, —C(O)C₁₋₁₀alkylcycloalkyl; —C(O)C₂₋₁₀alkenylcycloalkyl, —C(O)C₂₋₁₀alkynylcycloalkyl, —C(O)aryl, —C(O)C₁₋₁₀alkylaryl, —C(O)C₂₋₁₀alkenylaryl, —C(O)C₂₋₁₀alkynylaryl, —C(O)C₁₋₁₀alkylC(O)R₁₁, —C(O)C₂₋₂₀alkenylC(O)R₁₁, —C(O)C₂₋₁₀alkynylC(O)R₁₁, —C(O)C₁₋₁₀alkylCH(OR₁₁)(OR₁₁), —C(O)C₂₋₁₀alkenyl(CH)(OR₁₁)(OR₁₁), —C(O)C₂₋₁₀alkynylCH(OR₁₁)(OR₁₁), —C(O)C₁₋₁₀alkylSR₁₁, —C(O)C₂₋₁₀alkenylSR₁₁, —C(O)C₂₋₁₀alkynylSR₁₁, —C(O)C₁₋₁₀alkylC(O)OR₁₁, —C(O)C₂₋₁₀alkenylC(O)OR₁₁, —C(O)C₂₋₁₀alkynylC(O)OR₁₁, —C(O)C₁₋₁₀alkylC(O)SR₁₁, —C(O)C₂₋₁₀alkenylC(O)SR₁₁, —C(O)C₂₋₁₀alkynylC(O)SR₁₁,

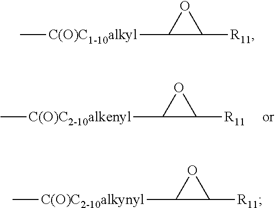

R₁₀ is —C₁₋₆alkyl, —C₂₋₆alkenyl, —C₂₋₆alkynyl, —C(O)C₁₋₆alkyl, —C(O)C₂₋₆alkenyl, —C(O)C₂₋₆alkynyl, —C(O)aryl, —C(O)C₁₋₆alkylaryl, —C(O)C₂₋₆alkenylaryl, —C(O)C₂₋₆alkynylaryl; and
R₁₁ is hydrogen, —C₁₋₁₀alkyl, —C₂₋₁₀alkenyl, —C₂₋₁₀alkynyl, cycloalkyl or aryl;
wherein each alkyl, alkenyl, alkynyl, cycloalkyl or aryl group is optionally substituted.

In some embodiments, the epoxytigliane compound of formula (I) is a compound of formula (II):

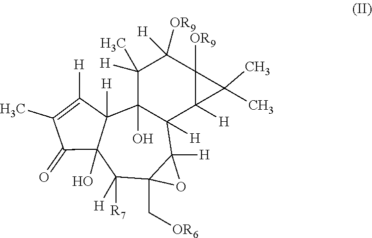

or a geometric isomer or stereoisomer or a pharmaceutically acceptable salt thereof; where R₆, R₇ and R₉ are as defined for formula (I).

In particular embodiments of formulae (I) or (II), one or more of the following applies:
R₁ is —C₁₋₃alkyl, especially —CH₃;
R₂ is —OC(O)C₁₋₂₀alkyl, —OC(O)C₂₋₂₀alkenyl, —OC(O)C₂₋₂₀alkynyl, —OC(O)cycloalkyl, —OC(O)C₁₋₁₀ alkylcycloalkyl; —OC(O)C₂₋₁₀alkenylcycloalkyl, —OC(O)C₂₋₁₀alkynylcycloalkyl, —OC(O)aryl, —OC(O)C₁₋₁₀alkylaryl, —OC(O)C₂₋₁₀alkenylaryl, —OC(O)C₂₋₁₀alkynylaryl, —OC(O)C₁₋₁₀alkylC(O)R₁₁, —OC(O)C₂₋₁₀alkenylC(O)R₁₁, —OC(O)C₂₋₁₀alkynylC(O)R₁₁, —OC(O)C₁₋₁₀alkylCH(OR₁₁)(OR₁₁), —OC(O)C₂₋₁₀ alkenylCH(OR₁₁)(OR₁₁), —OC(O)C₂₋₁₀alkynylCH(OR₁₁)(OR₁₁), —OC(O)C₁₋₁₀alkylSR₁₁, —OC(O)C₂₋₁₀alkenylSR₁₁, —OC(O)C₂₋₁₀alkynylSR₁₁, —OC(O)C₁₋₁₀alkylC(O)OR₁₁, —OC(O)C₂₋₁₀alkenylC(O)OR₁₁, —OC(O)C₂₋₁₀alkynylC(O)OR₁₁, —OC(O)C₁₋₁₀ alkylC(O)SR₁₁, —OC(O)C₂₋₁₀alkenylC(O)SR₁₁ or —OC(O)C₂₋₁₀alkynylC(O)SR₁₁; especially —OC(O)C₁₋₂₀alkyl, —OC(O)C₂₋₂₀alkenyl, —OC(O)C₂₋₂₀alkynyl, —OC(O)cycloalkyl, —OC(O)C₁₋₁₀alkylcycloalkyl; —OC(O)C₂₋₁₀alkenylcycloalkyl, —OC(O)C₂₋₁₀alkynylcycloalkyl or —OC(O)aryl; more especially —OC(O)C₁₋₂₀alkyl, —OC(O)C₂₋₂₀alkenyl or —OC(O)C₂₋₂₀alkynyl;
R₃ is —OC(O)C₁₋₂₀alkyl, —OC(O)C₂₋₂₀alkenyl, —OC(O)C₂₋₂₀alkynyl, —OC(O)cycloalkyl, —OC(O)C₁₋₁₀alkylcycloalkyl; —OC(O)C₂₋₁₀alkenylcycloalkyl, —OC(O)C₂₋₁₀alkynylcycloalkyl, —OC(O)aryl, —OC(O)C₁₋₁₀alkylaryl, —OC(O)C₂₋₁₀alkenylaryl, —OC(O)C₂₋₁₀alkynylaryl, —OC(O)C₁₋₁₀alkylC(O)R₁₁, —OC(O)C₂₋₁₀alkenylC(O)R₁₁, —OC(O)C₂₋₁₀alkynylC(O)R₁₁, —OC(O)C₁₋₁₀alkylCH(OR₁₁)(OR₁₁), —OC(O)C₂₋₁₀alkenylCH(OR₁₁)(OR₁₁), —OC(O)C₂₋₁₀alkynylCH(OR₁₁)(OR₁₁), —OC(O)C₁₋₁₀alkylSR₁₁, —OC(O)C₂₋₁₀alkenylSR₁₁, —OC(O)C₂₋₁₀alkynylSR₁₁, —OC(O)C₁₋₁₀alkylC(O)OR₁₁, —OC(O)C₂₋₁₀alkenylC(O)OR₁₁, —OC(O)C₂₋₁₀alkynylC(O)OR₁₁, —OC(O)C₁₋₁₀alkylC(O)SR₁₁, —OC(O)C₂₋₁₀alkenylC(O)SR₁₁ or —OC(O)C₂₋₁₀alkynylC(O)SR₁₁; especially —OC(O)C₁₋₂₀alkyl, —OC(O)C₂₋₂₀alkenyl, —OC(O)C₂₋₂₀alkynyl, —OC(O)cycloalkyl, —OC(O)C₁₋₁₀alkylcycloalkyl; —OC(O)C₂₋₁₀alkenylcycloalkyl, —OC(O)C₂₋₁₀alkynylcycloalkyl or —OC(O)aryl; more especially —OC(O)C₁₋₂₀alkyl, —OC(O)C₂₋₂₀alkenyl or —OC(O)C₂₋₂₀alkynyl;
R₄ and R₅ are independently selected from —C₁₋₃alkyl, especially —CH₃;
R₆ is hydrogen, —C(O)C₁₋₆alkyl, —C(O)C₂₋₆alkenyl, —C(O)C₂₋₆alkynyl or —C(O)aryl; especially hydrogen, —C(O)C₁₋₃alkyl, —C(O)C₂₋₃alkenyl or —C(O)C₂₋₃alkynyl, more especially hydrogen or —C(O)CH₃;
R₇ is hydroxyl, —OC(O)C₁₋₆alkyl, —OC(O)C₂₋₆alkenyl or —OC(O)C₂₋₆alkynyl, especially hydroxyl, —OC(O)C₁₋₃alkyl, —OC(O)C₂₋₃alkenyl or —OC(O)C₂₋₃alkynyl, more especially hydroxyl or —OC(O)CH₃; and
R₈ is —C₁₋₃alkyl, especially —CH₃.

In some embodiments, the epoxytigliane compound of formula (I) is a compound of formula (Ia):

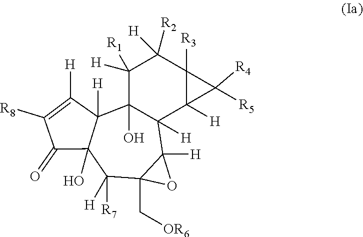

or a geometric isomer or stereoisomer or a pharmaceutically acceptable salt thereof,
wherein
   R₁ is hydrogen or C₁₋₆alkyl (e.g. R₁ is C₁₋₃alkyl, such as CH₃:
   R₂ is —OH or —OR₉;
   R₃ is —OH or —OR₉;
   R₄ and R₅ are independently selected from hydrogen and C₁₋₆alkyl (e.g. R₄ and R₅ are independently C₁₋₃alkyl, such as CH₃);
   R₆ is hydrogen or —R₁₀;
   R₇ is hydroxy or —OR₁₀;
   R₈ is hydrogen or C₁₋₆alkyl (e.g. R₈ is C₁₋₃alkyl, such as CH₃);
   R₉ is —C₁₋₂₀alkyl, —C₂₋₂₀alkenyl, —C₂₋₂₀alkynyl, —C(O)C₁₋₂₀alkyl, —C(O)C₂₋₂₀alkenyl, —C(O)C₂₋₂₀alkynyl, —C(O)C₃₋₆cycloalkyl, —C(O)C₁₋₁₀alkylC₃₋₆cycloalkyl; —C(O)C₂₋₁₀alkenylC₃₋₆cycloalkyl, —C(O)C₂₋₁₀alkynylC₃₋₆cycloalkyl, —C(O)aryl, —C(O)C₁₋₁₀alkylaryl, —C(O)C₂₋₁₀alkenylaryl, —C(O)C₂₋₁₀alkynylaryl, —C(O)C₁₋₁₀alkylC(O)R₁₁, —C(O)C₂₋₁₀alkenylC(O)R₁₁, —C(O)C₂₋₁₀alkynylC(O)R₁₁, —C(O)C₁₋₁₀alkylCH(OR₁₁)(OR₁₁), —C(O)C₂₋₁₀alkenylCH(OR₁₁)(OR₁₁), —C(O)C₂₋₁₀alkynylCH(OR₁₁)(OR₁₁), —C(O)C₁₋₁₀alkylSR₁₁, —C(O)C₂₋₁₀alkenylSR₁, —C(O)C₂₋₁₀alkynylSR₁₁, —C(O)C₁₋₁₀alkylC(O)OR₁₁, —C(O)C₂₋₁₀alkenylC(O)OR₁₁, —C(O)C₂₋₁₀alkynylC(O)OR₁₁, —C(O)C₁₋₁₀alkylC(O)SR₁₁, —C(O)C₂₋₁₀alkenylC(O)SR₁₁, —C(O)C₂₋₁₀alkynylC(O)SR₁₁,

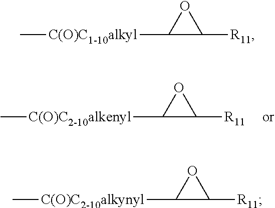

(e.g. R₉ is —C(O)C₁₋₂₀alkyl or —C(O)C₂₋₂₀alkenyl, such as tigloyl, 2-methylbutanoyl, hexanoyl, myristoyl, propanoyl or acetyl);
R₁₀ is —C₁₋₆alkyl, —C₂₋₆alkenyl, —C₂₋₆alkynyl, —C(O)C₁₋₆alkyl, —C(O)C₂₋₆alkenyl, —C(O)C₂₋₆alkynyl, —C(O)aryl, —C(O)C₁₋₆alkylaryl, —C(O)C₂₋₆alkenylaryl, or —C(O)C₂₋₆alkynylaryl (e.g. R₁₀is —C(O)C₁₋₃alkyl, such as —C(O)CH₃); and
R₁₁ is hydrogen, —C₁₋₁₀alkyl, —C₂₋₁₀alkenyl, —C₂₋₁₀alkynyl, C₃₋₆cycloalkyl or aryl.

In some embodiments, the epoxytigliane compound of formula (I) is a compound of formula (Ib):

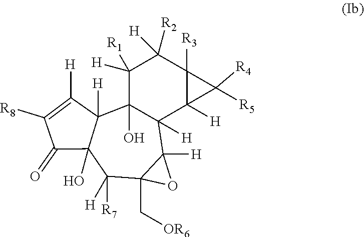

or a geometric isomer or stereoisomer or a pharmaceutically acceptable salt thereof; wherein
R₁ is C₁₋₃alkyl (e.g. R₁ is CH₃);
R₂ is —OR₉;
R₃ is —OR₉;
R₄ and R₅ are independently selected from C₁₋₃alkyl;
R₅ is hydrogen or —R₁₀;
R₇ is hydroxy;
R₈ is C₁₋₃alkyl;
R₉ is —C(O)C₁₋₂₀alkyl or —C(O)C₂₋₂₀alkenyl (e.g. R₉ is tigloyl, 2-methylbutanoyl, hexanoyl, myristoyl, propanoyl or acetyl);
R₁₀is —C(O)C₁₋₆alkyl (e.g. R₁₀ is —C(O)C₁₋₃alkyl, such as —C(O)CH₃).

In some embodiments, the compounds of formulae (I), (II), (Ia) and/or (Ib) have stereochemistry as shown in formula (IIII below:

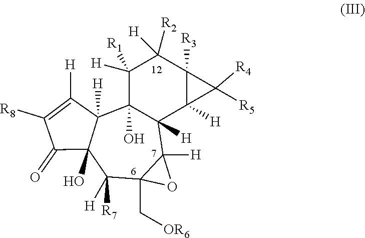

In some embodiments, the epoxide in the 6,7-position is above the plane of the ring system. In other embodiments, the epoxide in the 6,7-position is below the plane of the ring system. In some embodiments, the R₂ group in the 12 position is S and in other embodiments, the R₂ group in the 12 position is R.

In some embodiments, the epoxytigliane compound of formula (III) is a compound of formula (IIIa).

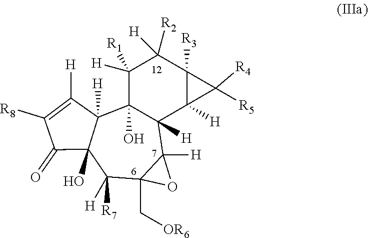

or a pharmaceutically acceptable salt thereof;
wherein
R₁ is selected from hydrogen and C₁₋₆alkyl (e.g. R₁ is C₁₋₃alkyl, such as CH₃);
R₂ is selected from —OC(O)C₁₋₇alkyl, —OC(O)C₂₋₇alkenyl and —OC(O)C₂₋₇alkynyl (e.g. R₂ is —O-tigloyl, —O-2-methylbutanoyl, —O-hexanoyl, —O-myristoyl or —O-propanoyl);
R₃ is selected from —OC(O)C₁₋₇alkyl, —OC(O)C₂₋₇alkenyl and —OC(O)C₂₋₇alkynyl (e.g. R₃ is —O-tigloyl, —O-2-methylbutanoyl, —O-hexanoyl, or —O-acetyl);
R₄ and R₅ are independently selected from hydrogen and C₁₋₆alkyl (e.g. R₄ and R₅ are independently C₁₋₃alkyl, such as CH₃);
R₆ is selected from hydrogen and —C(O)C₁₋₆alkyl (e.g. R₆ is —C(O)C₁₋₃alkyl, such as —C(O)CH₃);
R₇ is hydroxy; and
R₈ is selected from hydrogen or C₁₋₆alkyl (e.g. R₈ is C₁₋₃alkyl, such as CH₃).

In some embodiments, the epoxytigliane compound of formula (III) is a compound of formula (IIIb):

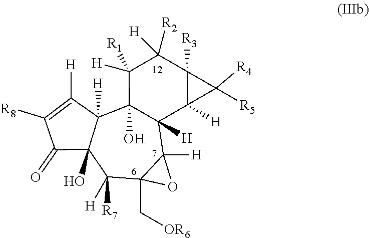

or a pharmaceutically acceptable salt thereof,
wherein
R₁ is selected from C₁₋₆alkyl (e.g. R₁ is C₁₋₃alkyl, such as CH₃);
R₂ is selected from —OC(O)C₁₋₇alkyl and —OC(O)C₂₋₇alkenyl (e.g. R₂ is —O-tigloyl, —O-2-methylbutanoyl, —O-hexanoyl, —O-myristoyl or —O-propanoyl);
R₃ is selected from —OC(O)C₁₋₇alkyl and —OC(O)C₂₋₇alkenyl (e.g. R₃ is —O-tigloyl, —O-2-methylbutanoyl, —O-hexanoyl, or —O-acetyl);
R₄ and R₅ are independently C₁₋₆alkyl (e.g. R₄ and R₅ are independently C₁₋₃alkyl, such as CH₃);
R₆ is selected from hydrogen and —C(O)C₁₋₆alkyl (e.g. R₆ is hydrogen or —C(O)C₁₋₃alkyl, such as —C(O)CH₃);
R₇ is hydroxy; and
R₈ is selected from C₁₋₆alkyl (e.g. R₈ is C₁₋₃alkyl, such as CH₃).

In particular embodiments the epoxytigliane compound is selected from:
12-tigloyl-13-(2-methylbutanoyl)-6,7-epoxy-4,5,9,12,13,20-hexahydroxy-1-tiglien-3-one (Compound 1);
12,13-di-(2-methylbutanoyl)-6,7-epoxy-4,5,9,12,13,20-hexahydroxy-1-tiglien-3-one (Compound 2);
12-hexanoyl-13-(2-methylbutanoyl)-6,7-epoxy-4,5,9,12,13,20-hexahydroxy-1-tiglien-3-one (Compound 3);
12,13-dihexanoyl-6,7-epoxy-4,5,9,12,13,20-hexahydroxy-1-tiglien-3-one (Compound 4);
12-myristoyl-13-(2-methylbutanoyl)-6,7-epoxy-4,5,9,12,13,20-hexahydroxy-1-tiglien-3-one (Compound 5);
12-tigloyl-13-(2-methylbutanoyl)-6,7-epoxy-4,5,9,12,13-pentahydroxy-20-acetyloxy-1-tiglien-3-one (Compound 6);
12-myristoyl-13-acetyloxy-6,7-epoxy-4,5,9,12,13,20-hexahydroxy-1-tiglien-3-one (Compound 7);
12-propanoyl-13-(2-methylbutanoyl)-6,7-epoxy-4,5,9,12,13,20-hexahydroxy-1-tiglien-3-one (Compound 8);
12,13-ditigloyl-6,7-epoxy-4,5,9,12,13,20-hexahydroxy-1-tiglien-3-one (Compound 9); and
12-(2-methylbutanoyl)-13-tigloyl-6,7-epoxy-4,5,9,12,13,20-hexahydroxy-1-tiglien-3-one (Compound 10).

In some embodiments, the epoxytigliane compound is tigilanol tiglate (abbreviated as “TT”), which is (4S,5S,6R,7S,8R,9R,10S,11R,12R,13S,14R)-12-(2E)-2-methyl-2-enoatyl-13((2S)-2-methylbutyroyl)-6,7-epoxy-4,5,9,12,13,20-hexahydroxy-1-tigliaen-3-one (which is also referred to as [(1R,2S,4R,5S,6S,10S,11R,12R,13R,14S,16R)-5,6,11-trihydroxy-4-(hydroxymethyl)-8,12,15,15-tetramethyl-13-[(E)-2-methylbut-2-enoyl]oxy-7-oxo-3-oxapentacyclo[9.5.0.0^{2,4}.0^{6,10}.0^{14,16}]hexadec-8-en-14-yl](2S)-2-methylbutanoate; chemical name computed by Lexichem TK 2.7.0 (PubChem release 2021.05.07), and which has the following structure:

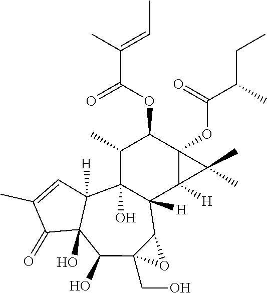

### Second Therapeutic Agent

The second therapeutic agent may be a cancer chemotherapeutic agent or irradiation. In some embodiments, the second therapeutic agent is a chemotherapeutic agent. In other embodiments, the second therapeutic agent is irradiation.

Without wishing to be bound by theory, it is postulated that the epoxytigliane compounds of formula (I) may synergise with cancer therapies that damage DNA or inhibit tumour-associated host-derived cells that support the growth and/or invasion of tumour cells. The second therapeutic agent is therefore one that damages DNA, resulting in cell death or one that kills tumour-associated host-derived cells, such as myeloid-derived suppressor cells (MDSC), which support the growth and/or invasion of tumour cells.

Examples of agents that damage DNA include DNA intercalating agents (which are substances that insert into the DNA structure of a cell and bind to the DNA to cause DNA damage) and DNA alkylating agents.

In some embodiments, the second therapeutic agent is a chemotherapeutic agent selected from:
i) A platinum chemotherapeutic agent, especially cisplatin and carboplatin, more especially cisplatin (Dasari and Tchounwou, 2013; Bracci et al., 2014);
ii) A DNA intercalating agent or inhibitor of the enzyme topoisomerase II, especially doxorubicin or mitoxantrone (Cheung-Ong et al. 2013);
iii) A DNA alkylating agent such as cyclophosphamide (Cheung-Ong et al. 2013; Hall and Tilby, 1992);
iv) An antimetabolite such as gemcitabine or fluorouracil, especially 5-fluorouracil (Cheung-Ong et al. 2013, Albeituni et al., 2013);
v) A vinca alkaloid such as vincristine (Moudi et al., 2013, Mohammadgholi et al. 2013); vi) A compound that binds to GC rich DNA such as bleomycin; and
vii) A MDSC inhibitor such as a phosphodiesterease-5 inhibitor, for example sildenafil; amiloride or a COX-2 and PEG-2 inhibitor such as celecoxib (Albeituni et al. 2013).

In some embodiments, the second therapeutic agent is, or comprises, cisplatin or carboplatin. In some embodiments, the second therapeutic agent is, or comprises, cisplatin.

In some embodiments, the second therapeutic agent is, or comprises, 5-fluorouracil.

In some embodiments, the second therapeutic agent is, or comprises, doxorubicin. Doxorubicin is considered to damage DNA through intercalation or direct alkylation and may result in the formation of DNA double-strand breaks.

In some embodiments, the second therapeutic agent is irradiation. Irradiation may for example occur by external beam radiation therapy (where high-energy beams are aimed at the site of the cancer from a machine outside the body) or by brachytherapy (where the beams come from a radiation source placed next to or inside the body). At high doses, radiation therapy (or ‘radiotherapy’) kills cancer cells and/or slows their growth by damaging their DNA. The radiation source may be any radioactive source typically used in cancer radiation therapy. In some embodiments, the radiation source is caesium (e.g. caesium¹³⁷). In some embodiments, the radiation source is cobalt (e.g. cobalt⁶⁰).

In some embodiments, the method of treatment comprises the use of an epoxytigliane compound (e.g. (4S,5S,6R,7S,8R,9R,10S,11R,12R,13S,14R)-12-(2E)-2-methyl-2-enoatyl-13((2S)-2-methylbutyroyl)-6,7-epoxy-4,5,9,12,13,20-hexahydroxy-1-tigliaen-3-one) and cisplatin. This combination may for example be used to treat squamous cell carcinoma (SCC), including in-situ SCC and invasive SCC (including UV-induced SCC).

In some embodiments, the method of treatment comprises the use of an epoxytigliane compound (e.g. (4S,5S,6R,7S,8R,9R,10S,11R,12R,13S,14R)-12-(2E)-2-methyl-2-enoatyl-13((2S)-2-methylbutyroyl)-6,7-epoxy-4,5,9,12,13,20-hexahydroxy-1-tigliaen-3-one) and 5-fluorouracil. This combination may for example be used to treat squamous cell carcinoma (SCC), including in-situ SCC and invasive SCC (including UV-induced SCC).

In some embodiments, the method of treatment comprises the use of an epoxytigliane compound (e.g. (4S,5S,6R,7S,8R,9R,10S,11R,12R,13S,14R)-12-(2E)-2-methyl-2-enoatyl-13((2S)-2-methylbutyroyl)-6,7-epoxy-4,5,9,12,13,20-hexahydroxy-1-tigliaen-3-one) and irradiation. This combination may for example be used to treat squamous cell carcinoma (SCC), including in-situ SCC and invasive SCC (including UV-induced SCC), and adenocarcinomas, including colonic adenocarcinomas.

In some embodiments, the method of treatment comprises the use of an epoxytigliane compound (e.g. (4S,5S,6R,7S,8R,9R,10S,11R,12R,13S,14R)-12-(2E)-2-methyl-2-enoatyl-13((2S)-2-methylbutyroyl)-6,7-epoxy-4,5,9,12,13,20-hexahydroxy-1-tigliaen-3-one) and doxorubicin. This combination may for example be used to treat melanoma and lung cancer.

In some embodiments, the method of treatment comprises the use of an epoxytigliane compound (e.g. (4S,5S,6R,7S,8R,9R,10S,11R,12R,13S,14R)-12-(2E)-2-methyl-2-enoatyl-13((2S)-2-methylbutyroyl)-6,7-epoxy-4,5,9,12,13,20-hexahydroxy-1-tigliaen-3-one) and irradiation.

The second chemotherapeutic agent is not an immune checkpoint inhibitor.

The epoxytigliane and the second therapeutic agent may be administered in accordance with any suitable dosing regimen. For example, they may be administered simultaneously, sequentially, or on different occasions.

In some embodiments, the therapeutic regime involves administration only of the two components, i.e. the epoxytigliane compound and the second therapeutic agent. For example, it may involve administration of the epoxytigliane compound and a chemotherapeutic agent that damages DNA only. Alternatively, it may involve administration of the epoxytigliane compound and a chemotherapeutic agent that inhibits tumour-associated host-derived cells that support the growth and/or invasion of tumour cells only. As a further alternative, the therapeutic regime may involve administration of the epoxytigliane compounds and irradiation only.

In some embodiments, the therapeutic regime involves administration of the epoxytigliane compound and only one second therapeutic agent, e.g. one chemotherapeutic agent that damages DNA, or one chemotherapeutic agent that inhibits tumour-associated host-derived cells that support the growth and/or invasion of tumour cells.

In some other embodiments, the therapeutic regime involves administration of the epoxytigliane compound and two or more of the second therapeutic agents (for example, two, three, four or five of the second therapeutic agents), e.g. it may involve administration of two chemotherapeutic agents that damage DNA, or two chemotherapeutic agents that inhibit tumour-associated host-derived cells that support the growth and/or invasion of tumour cells, or one of each of those two types of chemotherapeutic agents.

In some embodiments, the therapeutic regime does not include administration of any therapeutic agents other than the epoxytigliane compound and a second therapeutic agent. In some other embodiments, the therapeutic regime may involve administration of the epoxytigliane compound, a second therapeutic agent as defined herein, and one or more (e.g. one, two, three, four or five) further therapeutic agents.

### Compositions

While the epoxytigliane compounds or pharmaceutically acceptable salts thereof and second chemotherapeutic agent, may be administered neat, it may be more convenient to administer the epoxytigliane compounds and second chemotherapeutic agents in the form of one or more pharmaceutical compositions, each together with a pharmaceutically acceptable carrier, diluent and/or excipient.

Dosage form and rates for pharmaceutical use and compositions are readily determinable by a person of skill in the art.

In particular embodiments, the epoxytigliane compound is formulated for administration directly onto or into the tumour being treated. In some embodiments, the epoxytigliane compound is formulated for topical administration in the form of a gel, ointment, lotion, cream or transdermal patch that may be applied directly onto the tumour being treated. In other embodiments, the epoxytigliane compound is formulated for injection, especially intratumoural injection where the compound is injected into one or more places in a tumour.

The second chemotherapeutic agent may be administered in any means that is able to deliver the molecule systemically or locally. In some embodiments, the second chemotherapeutic agent is conveniently delivered by injection, for example, intravenous, intraarticular, intramuscular, intradermal, subcutaneous or intraperitoneal injection. The second chemotherapeutic agent may also be formulated for local delivery by injection, for example, intratumourally. Pharmaceutically acceptable carriers and acceptable carriers for systemic or local administration may also be incorporated into the compositions of the second chemotherapeutic agents.

In some embodiments, the epoxytigliane compound and the second chemotherapeutic agent are delivered separately, either simultaneously or sequentially. In other embodiments, the epoxytigliane compound and the second chemotherapeutic agent are delivered in a single composition, for example, a single composition suitable for intratumoural delivery or a single composition formulated for systemic delivery.

In another aspect, there is provided a pharmaceutical composition comprising an epoxytigliane compound or a pharmaceutically acceptable salt thereof and a second chemotherapeutic agent, optionally together with one or more pharmaceutically acceptable carriers.

Suitably, the pharmaceutical composition(s) comprise a pharmaceutically acceptable excipient or an acceptable excipient. By “pharmaceutically acceptable excipient” is meant a solid or liquid filler, diluent or encapsulating substance that may be safely used. Depending upon the particular route of administration, a variety of carriers, well known in the art may be used. These carriers or excipients may be selected from a group including sugars, starches, cellulose and its derivatives, cyclodextrins, malt, gelatine or other gelling agents, polymers, talc, calcium sulphate, vegetable oils, synthetic oils, alcohols and/or polyols, alginic acid, phosphate buffered solutions, emulsifiers, isotonic saline, and pyrogen-free water.

Liquid form preparations include solutions, suspensions, and emulsions, for example, water or water-propylene glycol solutions. For example, parenteral injection liquid preparations can be formulated as solutions in aqueous 1,2-propanediol, dimethylsulfoxide (DMSO), aqueous solutions of gamma cyclodextrin or 2-hydroxypropyl-beta-cyclodextrin, saline solution or polyethylene glycol solution, with or without buffer. A preferred range of pH is 3.0-4.5. Suitable buffers buffer the preparation at pH 3.5-4.5 and include, but are not limited to, acetate buffer and citrate buffer.

The compositions of epoxytigliane compound and/or second chemotherapeutic agent may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, gels or emulsions in oily or aqueous vehicles, and may contain formulatory agents such as suspending, stabilising and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

Pharmaceutical compositions of epoxytigliane compound and/or second chemotherapeutic agent suitable for administration may be presented in discrete units such as syringes, vials, tubes or sachets each containing a predetermined amount of one or more pharmaceutically active compounds or extracts of the invention, as a powder or granules or as a solution or a suspension in an aqueous liquid, a cyclodextrin solution, a non-aqueous liquid, an oil-in-water emulsion or a water-in-oil emulsion or as a solution or suspension in a cream or gel or as a suspension of micro- or nano-particles incorporating an epoxytigliane compound, including but not limited to silica or polylactide micro- or nano-particles. Such compositions may be prepared by any of the method of pharmacy but all methods include the step of bringing into association one or more pharmaceutically active compounds of the invention with the carrier which constitutes one or more necessary ingredients. In general, the compositions are prepared by uniformly and intimately admixing the agents of the invention with liquid carriers or finely divided solid carriers or both, and then, if necessary, shaping the product in to the desired presentation.

For topical administration to the epidermis or other organ, the epoxytigliane compound and/or second chemotherapeutic agent may be formulated as gels, ointments, emulsions, pastes, creams or lotions, or as a transdermal patch. Gels may be prepared using suitable thickening agents and adding them to aqueous/alcoholic compositions of compound. Suitable thickening or gelling agents are known in the art, such as the polyvinyl carboxy polymer Carbomer 940. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Formulations suitable for topical administration also include solutions or suspensions that may be administered topically in the form of a bath or soak solution or a spray or may be absorbed into a dressing.

When the second chemotherapeutic agent is a small molecule, it may be delivered by any suitable means including oral, topical, rectal, parenteral, sublingual, buccal, intravenous, intraarticular, intra-muscular, intra-dermal, subcutaneous, inhalational, intraocular, intraperitoneal, intracerebroventricular, transdermal and the like as known in the art of pharmacy.

### Dosage Regimens

In some embodiments, the epoxytigliane compound is delivered in the same composition as the second chemotherapeutic agent. However, in particular embodiments, the second chemotherapeutic agent is administered in a separate composition from the epoxytigliane compound. When the second therapeutic agent is irradiation, this is of course, delivered separately from the administration of the epoxytigliane compound.

In some embodiments, the epoxytigliane compound is administered directly to the tumour, for example by topical administration or by intra-tumoural injection.

In some embodiments, the epoxytigliane compound is administered to the tumour once. In other embodiments, the treated tumour is monitored and further administration of epoxytigliane compound may be required if the tumour does not fully respond to the treatment. In embodiments where the tumour is treated topically, the epoxytigliane compound may be administered on a number of occasions over a period of time, for example, daily for a week, or once a week for 4 to 10 weeks. A person skilled in the art, monitoring the subject being treated would be able to determine an appropriate dosage schedule, which may vary depending on the response to the treatment.

In particular embodiments, the epoxytigliane compound is administered once only by intratumoural injection.

In some embodiments, the amount of epoxytigliane compound delivered is a sub-therapeutic amount.

In some embodiments, the second chemotherapeutic agent is administered at least once, prior to or simultaneously or sequentially with the epoxytigliane compound. In some embodiments the second chemotherapeutic agent is administered once, either before, concurrently with or after the administration of the epoxytigliane compound. For example, in some embodiments, the second chemotherapeutic agent is administered within the 24 hours before, immediately before, concurrently with, immediately after or within 24 hours after the administration of the epoxytigliane compound, especially immediately before, concurrently with and immediately after the administration of the epoxytigliane. By immediately before or immediately after is meant that the administration of the second chemotherapeutic agent occurs less than 2 hours before or after the administration of the epoxytigliane compound, especially less than one hour and more especially less than 30 minutes.

In some embodiments, multiple doses of the second chemotherapeutic agent are administered over a period of time beginning before or together with administration of the epoxytigliane compound and then continuing after administration of the epoxytigliane compound. In some embodiments, the second chemotherapeutic agent is administered more than once and on a regular basis before and after administration of the epoxytigliane compound. In particular embodiments, the second chemotherapeutic agent is administered before administration of the epoxytigliane compound, sequentially or simultaneously with the administration of the epoxytigliane compound and at least once subsequently to administration of the epoxytigliane compound. For example, the second chemotherapeutic agent may be administered 24 hours prior to administration of the epoxytigliane compound, the second chemotherapeutic agent is then administered sequentially or simultaneously with the epoxytigliane compound, either immediately before or immediately after the administration of the epoxytigliane compound, the second chemotherapeutic agent is then administered one or more times over the next one to three months after administration of the epoxytigliane compound, for example, once a week, once every 5 days, once every 4 days, once every 3 days, every 2 days or every day, especially every 1 to 3 days, more especially every 2 days. Subsequent administration of the second chemotherapeutic agent may continue such that 1 to 10 doses of second chemotherapeutic agent are administered after the administration of the epoxytigliane compound, especially 1 to 8 doses, 1 to 6 doses, 1 to 4 doses, 1 to 3 doses or 1 to 2 doses.

In some embodiments, the epoxytigliane compound is administered in an effective amount. An “effective amount” means an amount necessary at least partly to attain the desired response, for example, to reduce the size of the tumour or to destroy the tumour in total. The amount varies depending upon the health and physical condition of the individual to be treated, the taxonomic group of individual to be treated, the formulation of the composition, the size of the tumour, the assessment of the medical situation, the second therapeutic agent it is combined with and other relevant factors. It is expected that the amount will fall in a relatively broad range that can be determined through routine trials. An effective amount, for example, may lie in the range of about 0.1 μg per cm³ tumour volume to 20 mg per cm³ tumour volume per dosage. The dosage is preferably in the range of 1 μg to 10 mg per cm³ tumour volume per dosage, such as is in the range of 0.5 μg to 5 mg per cm³ tumour volume per dosage. In one embodiment, where the dosage is administered intra-tumourally, the dosage is in the range of 0.01 to 1 mg per cm³ tumour volume, for example 0.1 mg to 1 mg per cm³ tumour volume to 0.7 mg per cm³ tumour volume, 0.1 to 0.8 mg per cm³ tumour volume, such as 0.125 mg to 0.7 mg per cm³ tumour volume. In another embodiment, the dosage is in the range of 0.001 mg to 20 mg per dosage, for example, 0.005 mg to 15 mg per dosage, especially 0.05 to 10 mg per dosage, more especially about 0.1 to about 5 mg per dosage. Dosage regimes may be adjusted to provide the optimum therapeutic response. For example, in some embodiments, where administration is intra-tumoural, the epoxytigliane compound is administered once and the progress of treatment monitored. In some embodiments, if the tumour does not completely resolve or if the tumour recurs, a second dose may be administered. In some embodiments, where the administration is topical, the topical compound formulation may be administered directly onto the site of the tumour in the form of a gel, cream, ointment or lotion. The frequency of treatment will depend on the tumour, its size, the subject being treated and the like. In some embodiments, a topical formulation may be applied weekly until the tumour is resolved. In other embodiments, the treatment may be a single treatment and a second treatment only administered if the tumour is not completely resolved.

In some embodiments the epoxytigliane compound is administered in a sub-therapeutic amount. A sub-therapeutic amount is an amount that is less than an amount expected to be effective if the epoxytigliane compound was administered alone. For example, the sub-therapeutic amount may be at the lower end of the effective amount described above for example, 0.1 to 0.2 mg per cm³ tumour volume.

The second chemotherapeutic agent may also be administered in an effective amount. Again, the amount of second chemotherapeutic agent considered to be effective will depend on the subject being treated, their health and physical condition, the identity of the second chemotherapeutic agent, the formulation of the composition and the assessment of the medical situation. It is expected that the amount of the second chemotherapeutic agent will fall within a fairly broad range of amounts. An effective amount may lie in the range of about 0.1 ng per kg to about 500 mg per kg body weight, 100 μg per kg to 100 mg per kg body weight, 1 mg per kg to 50 mg per kg body weight, 1 mg per kg to 20 mg per kg body weight. In another embodiment, the actual dosages may be in the range of from 1 μg to 1 g, for example, 100 μg to 750 mg per dose. The effective amount may be a sub-therapeutic amount when used as a monotherapy but when in combination with the epoxytigliane compound is an effective amount.

When the second therapeutic agent is irradiation, for example external beam radiation, the radiation may be administered at least once. In some embodiments, the radiation is administered just once either within 24 hours before, immediately before, immediately after or within 24 hours after administration of the epoxytigliane compound, especially immediately before or immediately after administration of the epoxytigliane compound. By immediately before or immediately after is meant that the irradiation occurs less than 2 hours before or after the administration of the epoxytigliane compound, especially less than one hour and more especially less than 30 minutes.

In some embodiments, the radiation is administered more than once, for example, within the irradiation may be administered 24 hours prior to administration of the epoxytigliane compound, then administered sequentially or simultaneously with the epoxytigliane compound, either immediately before or immediately after the administration of the epoxytigliane compound, then administered one or more times over the next month after administration of the epoxytigliane compound, for example, once a week, once every 5 days, once every 4 days, once every 3 days, every 2 days or every day.

In some embodiments, the amount of irradiation administered is between 1 and 8 Gray (Gy) per dose. In particular embodiments, the amount of irradiation administered is between 1 and 6 Gy, 2 and 4 Gy or about 2 Gy per dose, for example to deliver a total dose between 8 and 50 Gy. In some embodiments, irradiation is administered once at a sub-optimal dose, for example, between 2 and 8 Gy total dose, especially 2 to 4 Gy or 1 to 2 Gy.

The subject that may be treated with the combination therapy is a mammal, a bird, an aquatic animal such as a fish, or a reptile. In some embodiments, the subject is a human, a laboratory animal such as a mouse, rat or rabbit, a companion animal such as a dog or cat, a working animal such as a horse, donkey and the like, a livestock animal such as a cow, bull, pig, sheep, goat, deer, llama, alpaca and the like, or a captive wild animal such as those in zoos or wildlife parks including lions, leopards, cheetah, elephant, zebra, antelope, giraffe, koala, kangaroo and reptiles such as crocodiles, lizards, snakes and the like, a bird, especially a captive bird, such as a budgerigar or canary, cockatoo, parakeet, macaw, parrot and the like, or a fish, especially a captive fish such as tropical fish (zebra fish, guppy, Siamese fighting fish, clown fish, cardinal tetra and the like), dolphins, whales, and the like. In particular embodiments, the subject is a human or a companion animal.

In some embodiments, the subject is immunocompetent. In other embodiments, the subject is immunosuppressed or immunocompromised.

### Kits

The compositions of epoxytigliane compound and the second chemotherapeutic agent may be formulated separately and sold together in a kit or package. Each kit may comprise dosages of each compound to achieve treatment of a tumour.

In some embodiments, the epoxytigliane composition is formulated for topical administration, such as in a gel, lotion, cream or ointment or is impregnated into a dressing. In other embodiments, the epoxytigliane compound is formulated for injection such as intratumoural injection. In this embodiment, the epoxytigliane formulation may be present in the kit as a liquid ready for uptake into a syringe, as a powder or solid formulation which may be solubilized in a carrier before injection or may be present in the kit in a pre-filled syringe.

Each kit may comprise one or more doses of epoxytigliane compound. In one embodiment, the kit will contain a single dose of epoxytigliane compound in a formulation suitable for intratumoural injection. In another embodiment, the kit will contain a topical formulation of epoxytigliane compound containing multiple doses for application to the tumour.

In some embodiments, the second chemotherapeutic agent is formulated for parenteral administration in a single bolus dose or in a multiple dose form. For example, the kit may contain the second chemotherapeutic agent in a pre-filled syringe, as a liquid in a vial ready for uptake into a syringe, or as a solid ready for dissolution before uptake into a syringe. The liquid or solid formulations may be single dose formulations or multiple dose formulations. Alternatively, the kit may contain multiple doses of second chemotherapeutic agent, each formulated separately in a prefilled syringe, as a liquid in a vial ready for uptake into a syringe or as a solid ready for dissolution and uptake into a syringe.

The kit may further comprise an insert with instruction for use of each formulation, including how to prepare each dose if required, how to administer each dosage and when to administer each dosage.

In some embodiments, the kit comprises an epoxytigliane compound (e.g. (4S,5S,6R,7S,8R,9R,10S,11R,12R,13S,14R)-12-(2E)-2-methyl-2-enoatyl-13((2S)-2-methylbutyroyl)-6,7-epoxy-4,5,9,12,13,20-hexahydroxy-1-tigliaen-3-one), which may for example be for use with irradiation to treat tumours such as squamous cell carcinoma (SCC), including in-situ SCC and invasive SCC, and adenocarcinomas, including colonic adenocarcinomas.

In some embodiments, the kit comprises an epoxytigliane compound (e.g. (4S,5S,6R,7S,8R,9R,10S,11R,12R,13S,14R)-12-(2E)-2-methyl-2-enoatyl-13((2S)-2-methylbutyroyl)-6,7-epoxy-4,5,9,12,13,20-hexahydroxy-1-tigliaen-3-one) and cisplatin. This kit may for example be used to treat squamous cell carcinoma (SCC), including in-situ SCC and invasive SCC.

In some embodiments, the kit comprises an epoxytigliane compound (e.g. (4S,5S,6R,7S,8R,9R,10S,11R,12R,13S,14R)-12-(2E)-2-methyl-2-enoatyl-13((2S)-2-methylbutyroyl)-6,7-epoxy-4,5,9,12,13,20-hexahydroxy-1-tigliaen-3-one) and 5-fluorouracil.

This kit may for example be used to treat squamous cell carcinoma (SCC), including in-situ SCC and invasive SCC.

In some embodiments, the kit comprises an epoxytigliane compound (e.g. (4S,5S,6R,7S,8R,9R,10S,11R,12R,13S,14R)-12-(2E)-2-methyl-2-enoatyl-13((2S)-2-methylbutyroyl)-6,7-epoxy-4,5,9,12,13,20-hexahydroxy-1-tigliaen-3-one) and doxorubicin. This kit may for example be used to treat melanoma and lung cancer.

The present disclosure is further illustrated by the following non-limiting examples.

### EXAMPLES

### Example 1: In Vivo Combinations of Compound 1 and Cisplatin

Prior to commencement of the in vivo experiment, ethics approval was obtained from the Queensland Institute of Medical Research (QIMR) Animal Ethics Committee. All mice were house in pathogen-free conditions in the QIMR Animal Facility where they were caged with access to filtered air, food and water were freely available. Mice were handled and procedures performed using aseptic techniques within a laminar flow hood.

Prior to inoculation of subject mice, the cancer cells were seeded to T175 tissue culture flasks (Thermo Scientific) to increase cell numbers. Once 70-80% confluent, cells were trypsinized and resuspended in RPMI-1640 media for harvesting and cell count using a haemocytometer. The cell volume was centrifuged and the pellet reconstituted in RPMI-1640 media to a volume sufficient for the number of inoculations required at 100 μL per injection with two sites per mouse subject. The cell suspension was kept on ice and transferred to the animal facility for injection into the subjects.

The details of the mice used and the cell lines used for the combination experiment is provided in Table 1. The BALB/c Foxn1^{nu} mice were obtained from the Animal Resource Centre, Western Australia. The C3H/HeNCr mice were bred at the QIMR Animal Facility.

TABLE 1

| Model | Mouse | Age | | Gender | Cell Line | Cell volume |
|---|---|---|---|---|---|---|
| Syngeneic | C3H/HeNCr | 4-18 | weeks | Mixed | UV-13-1 | 1 × 10⁶ cells |
| Xenogeneic | BALB/c | 4 | weeks | Male | SCC15 | 1 × 10⁶ cells |

Foxn1^{nu}

C3H/HeNCr mice were shaved (Wahl, Illinois, USA) one day before inoculation. Whilst restrained subjects were inoculated on the hind quarter bilaterally by injecting 100 μL of the appropriate cell suspension into the subcutaneous space with a Terumo® (Tokyo, Japan) 27G×½″ needle. Subjects were ear tagged for identification. Following inoculation, mice were monitored twice weekly for tumour development and well-being and once tumours developed, the subjects were monitored daily.

A cohort of thirty BALB/c Foxn1^{nu} and twenty-one C3H/HeNCr mice, each with two tumours per mouse were divided into six experimental groups as set out in Table 2. 30 μg Compound 1 is considered a therapeutic dose for a 100 mm³ tumour so 5 μg and 10 μg were selected to be sub-optimal doses.

TABLE 2

Cisplatin Intraperitoneal (IP)

| | | | | |
|---|---|---|---|---|
| Compound 1 | 0 | μg | 0 mg/kg | 2.5 mg/kg |
| Intratumoural (IT) | 5 | μg | 0 mg/kg | 2.5 mg/kg |
| | 10 | μg | 0 mg/kg | 2.5 mg/kg |

Weight was recorded for each subject prior to and post-treatment, and at the time of culling. The width and length of each tumour was measured using 150 mm digital Vernier callipers (Kinchrome, Qld, AU) and individual tumour volume (mm³) calculated using the formula:

$$( length \times ( {width}^{2} ) ) \div 2$$

Twenty-four hours before it was anticipated that tumours would reach a volume of 70-100 mm³, the test subjects were administered either 2.5 mg/kg cisplatin (0.5 mg/mL in 0.9% normal saline) or an equivalent volume of 0.9% normal saline intraperitoneally using a Terumo® 0.5 mL U-100 insulin 29 G×½″ needle. IP injections were performed by first sterilizing the anterior abdominal wall of the subject with an alcohol swab before the dorsal skin of the subject was scruffed and the subject inverted with head down. A 29 G needle with the bevel upwards is used to inject into the lower right abdominal quadrant.

On reaching a volume of 70-100 mm³, tumours of the test subjects were injected IT with 50 μL of either 5 μg or 10 μg of Compound 1 or for control subjects 50 μL of 40% propylene glycol (PG).

Test subjects received further IP injections of cisplatin and 0.9% normal saline at day 7 and day 14 following initial injections. Compound 1 concentration was confirmed by high performance liquid chromatography (HPLC) prior to use.

Test subjects were observed daily and monitored for up to three months following treatment. Tumour volume was recorded as above.

The subjects were euthanized by CO₂ inhalation when total tumour burden reached 1000 mm³ per mouse, if ill, and on completion of the experiment.

Data was analysed with Microsoft Excel and Prism 7 to calculate average tumour volumes and standard deviations (SD). Individual and averaged tumour volumes and Kaplan-Meier survival curves were graphed relative to time of treatment.

### Xenogeneic Model

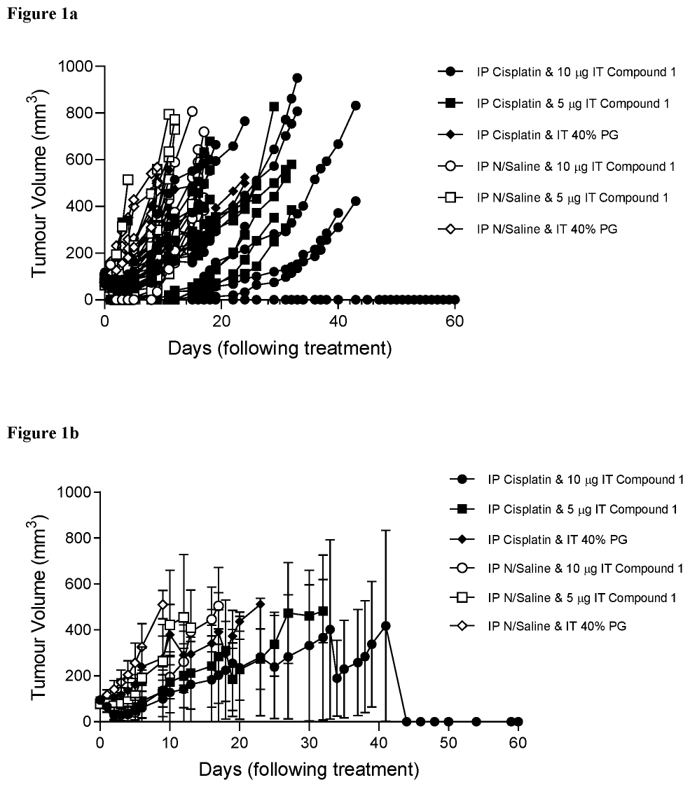

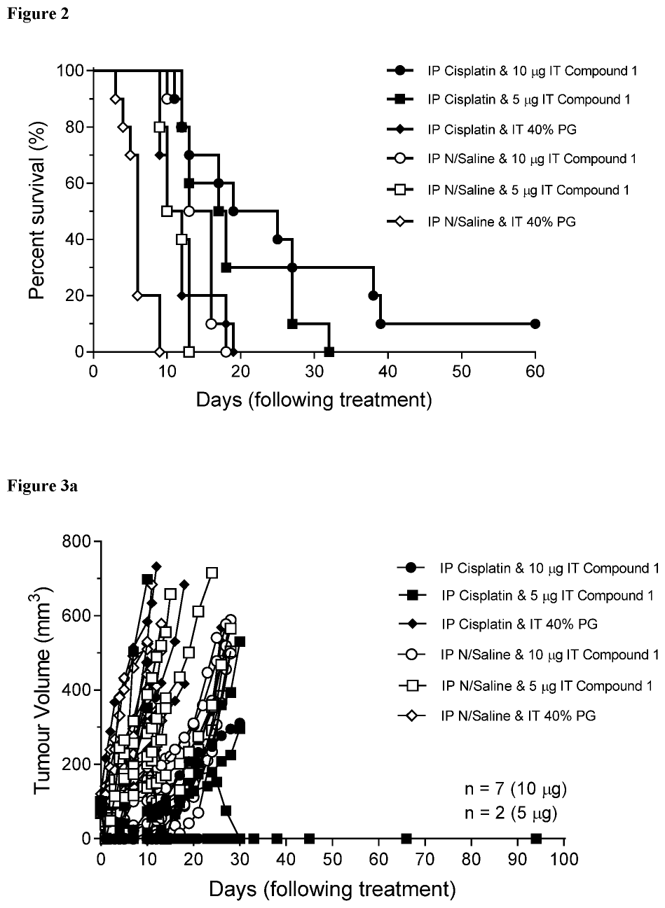

Individual SCC-15 tumour volumes and average tumour volumes for the combination of Compound 1 and cisplatin in the BALB/c Foxn1^{nu} mouse model are shown in FIGS. 1a and 1b respectively. Kaplan-Meier survival curves for the xenogeneic model are shown in FIG. 2.

The BALB/c Foxn1^{nu} mouse model is an immunocompromised host that lacks an intact acquired immune system. This model allows assessment of the Compound 1/cisplatin combination therapy where the host immune system is less likely to be contributing to any treatment effect.

From FIGS. 1a/b and 2, there is a clear trend across individual and average tumour volumes, and the Kaplan-Meier survival curves that combination therapy has superior efficacy, particularly at the higher concentration of Compound 1—2.5 mg/kg cisplatin IP and 10 μg Compound 1 IT. This combination resulted in a long-lasting response with one tumour remaining cured at 60 days. Lower dose (5 μg) of Compound 1 was not as effective as the higher dose but still demonstrated superior efficacy to that of single agent therapy or no treatment.

Subject weight (g) served as a marker for systemic toxicity of IP cisplatin. This allowed assessment of whether combination therapy with sub-therapeutic concentrations of IP cisplatin demonstrate efficacy whilst avoiding the usual side effects associated with its administration. The results are shown in Table 3.

TABLE 3

| | |
|---|---|
| IP N/Saline | IP Cisplatin |

| Time Point | Average | SD | Average | SD |
|---|---|---|---|---|
| Pre-treatment | 18.95 | 0.82 | 20.53 | 1.90 |
| Post-treatment | 19.24 | 0.89 | 20.44 | 1.85 |
| Cull | 21.23 | 0.91 | 22.68 | 2.19 |

The results show that there is very little weight difference pre- and post-treatment, but by the end of the experiment there is a clear weight gain for both the control and cisplatin treated subjects. Although a small weight loss was observed post-treatment with cisplatin, the weight was recovered by the end of the experiment. While the weight gain in both saline treated and cisplatin treated from pre-treatment to end of experiment was statistically significant, the weight loss from pre-treatment to post-treatment with cisplatin was not statistically significant.

### Syngeneic Model

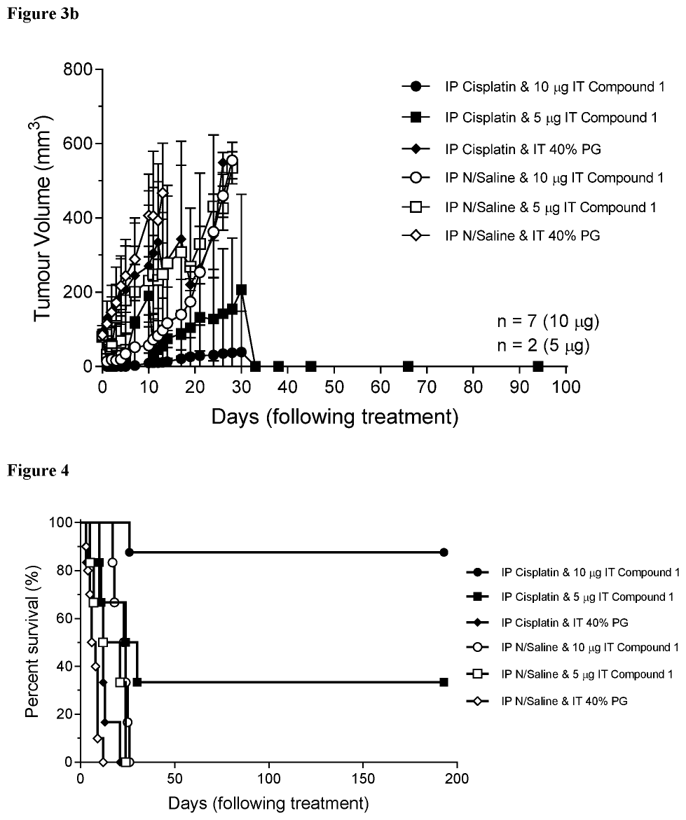

Individual UV-13-1 tumour volumes and average tumour volumes for the combination of Compound 1 and cisplatin in the C3H/HNCr syngeneic mouse model are shown in FIGS. 3a and 3b respectively. Kaplan-Meier survival curves for the syngeneic mouse model are shown in FIG. 4.

C3H/HeNCr mouse model inoculated with UV-13-1 tumours is a syngeneic model where the mouse is immunocompetent and the influence of the immune system on treatment may be assessed. These mice were not culled at 60 days but were periodically (weekly) monitored up to 200 days for recurrence.

Similarly to the xenogeneic model, it is clear that the combination therapy of Compound 1 IT and IP cisplatin is the most efficacious of the therapies demonstrating greatly improved individual and average tumour sizes as well as improved long term survival. FIG. 4 demonstrates that the combination therapy leads to the best duration of response.

Initial response to treatment with 10 μg Compound 1 and saline is equivalent to the combination of Compound 1 and cisplatin. However, at day 18, the monotherapy with Compound 1 falls away in efficacy significantly. The average tumour sizes also reflect this trend. Only the combination of Compound 1 and cisplatin resulted in a durable tumour response with several subjects exhibiting no evidence of recurrence at experiment end. Both 5 μg and 10 μg of Compound 1 were more efficacious than IP cisplatin alone.

The results of the combination in the syngeneic model are more marked and durable than those in the xenogeneic model. Only one tumour remained cured in the xenogeneic model at 60 days for the combination therapy, but at 200 days post-treatment in the syngeneic model, 9 tumours remained cured.

In the syngeneic model weight gain was also used as a marker of cisplatin toxicity. The results are shown in Table 4.

TABLE 4

| | |
|---|---|
| IP N/Saline | IP Cisplatin |

| Time Point | Average | SD | Average | SD |
|---|---|---|---|---|
| Pre-treatment | 26.71 | 6.0 | 26.16 | 4.00 |
| Post-treatment | 26.71 | 5.60 | 25.59 | 4.13 |
| Cull | 28.08 | 4.62 | 27.20 | 2.81 |

The results show that the change in weight over the course of the experiment in the saline control was not statistically significant. While the change in weight pre- and post-treatment with the IP cisplatin group was statistically significant (p=<0.0001), the weight gain from pre-treatment to death in this group was not significant. The statistically significant drop in weight from pre-treatment to post-treatment indicates that even the use of sub-therapeutic concentrations of cisplatin has some toxicity.

### Summary

A summary of survival with each treatment is shown in Table 5.

TABLE 5

| | Median Survival (days) | |
|---|---|---|
| Treatment | Xenogeneic Model | Syngeneic Model |
| IP N/Saline + IT 40% PG | 6 | 7 |
| IP N/Saline + IT 5 μg | 11 | 16.5 |
| Compound 1 | | |
| IP N/Saline + IT 10 μg | 13 | 23.5 |
| Compound 1 | | |
| IP cisplatin + IT 40% PG | 11 | 12 |
| IP cisplatin + IT 5 μg | 17.5 | 30 |
| Compound 1 | | |
| IP cisplatin + IT 10 μg | 22 | 193 |
| Compound 1 | | |

### Example 2: In Vivo Combination of Compound 1 and Irradiation

Prior to commencement of the in vivo experiment, ethics approval was obtained from the Queensland Institute of Medical Research (QIMR) Animal Ethics Committee. All mice were housed in pathogen-free conditions in the QIMR Animal Facility where they were caged with access to filtered air, food and water were freely available. Mice were handled and procedures performed using aseptic techniques within a laminar flow hood.

Prior to inoculation of subject mice, the cancer cells were seeded to T175 tissue culture flasks (Thermo Scientific) to increase cell numbers. Once 70-80% confluent, cells were trypsinized and resuspended in RPMI-1640 media for harvesting and cell count using a haemocytometer. The cell volume was centrifuged and the pellet reconstituted in RPMI-1640 media to a volume sufficient for the number of inoculations required at 100 μL per injection with two sites per mouse subject. The cell suspension was kept on ice and transferred to the animal facility for injection into the subjects.

The details of the mice used and the cell lines used for the combination experiment is provided in Table 6. The BALB/c Foxn1^{nu} and C57BL/6 mice were obtained from the Animal Resource Centre, Western Australia.

TABLE 6

| Model | Mouse | Age | Gender | Cell Line | Cell volume |
|---|---|---|---|---|---|
| Syngeneic | C57BL/6 | 6 weeks | Male | MC38 | 1 × 10⁶ cells |
| Xenogeneic | BALB/c | 4 weeks | Male | SCC-15 | 1 × 10⁶ cells |
| | Foxn1^{nu} | | | | |

The MC38 cell line is a murine colonic adenocarcinoma cell line derived from the C57BL/6 mouse (Kerafast, ENH204).

C57BL/6 mice were shaved (Wahl, Illinois, USA) one day before inoculation. Whilst restrained subjects were inoculated on the hind quarter bilaterally by injecting 100 μL of the appropriate cell suspension into the subcutaneous space with a Terumo® (Tokyo, Japan) 27G×½″needle. Subjects were ear tagged for identification. Following inoculation, mice were monitored twice weekly for tumour development and well-being and once tumours developed, the subjects were monitored daily.

A cohort of thirty BALB/c Foxn1^{nu} and thirty C56BL/6 mice, each with two tumours per mouse were divided into six experimental groups as set out in Table 7. 30 μg Compound 1 is considered a therapeutic dose, 7.5 μg was selected to be a suboptimal dose.

TABLE 7

Compound 1, Intratumoural (IT)

| | | | | |
|---|---|---|---|---|
| Irradiation (Gy) | 0 | Gy | 0 μg | 7.5 μg |
| | 5 | Gy | 0 μg | 7.5 μg |
| | 10 | Gy | 0 μg | 7.5 μg |

The width and length of each tumour was measured using 150 mm digital Vernier callipers (Kinchrome, Qld, AU) and individual tumour volume (mm³) calculated using the formula:

(length×(width²))÷2

On reaching a volume of 70-100 mm³, tumours of the test subjects were irradiated with 0, 5 or 10 Gy. Adequate positioning was ensured with the irradiator (Gammacel 40 Extractor) by sedating each mouse with a combination solution of Ketamine (80 mg/kg of 8 mg/mL solution, Ceva, NSW, AU) and Midazolam (4 mg/kg of 0.4 mg/mL solution, Sandoz, Hozkirchen, Germany) via intraperitoneal injection. Mice were double contained during transfer to and from the irradiator. During irradiation, mice were restrained with lead shielding to prevent exposure to sites other than the flanks where the tumours were located. Immediately upon return to the Animal Facility, mice received intraturmoral (IT) injections with 50 μL of 7.5 μg of Compound 1 or for control subjects 50 μL of 40% propylene glycol (PG) using a Terumo 0.5 mL U-100 insulin 29G×½″ needle. On completion of treatment, mice were warmed with a heating blanket, placed on their side, and observed until ambulant.

Test subjects were observed daily and monitored for up to three months following treatment. Tumour volume was recorded as above.

The subjects were euthanized by CO₂ inhalation when total tumour burden reached 1000 mm³ per mouse, if ill, and on completion of the experiment.

Data was analysed with Microsoft Excel and Prism 7 to calculate average tumour volumes and standard deviations (SD). Individual and averaged tumour volumes and Kaplan-Meier survival curves were graphed relative to time of treatment.

### Xenogeneic Model

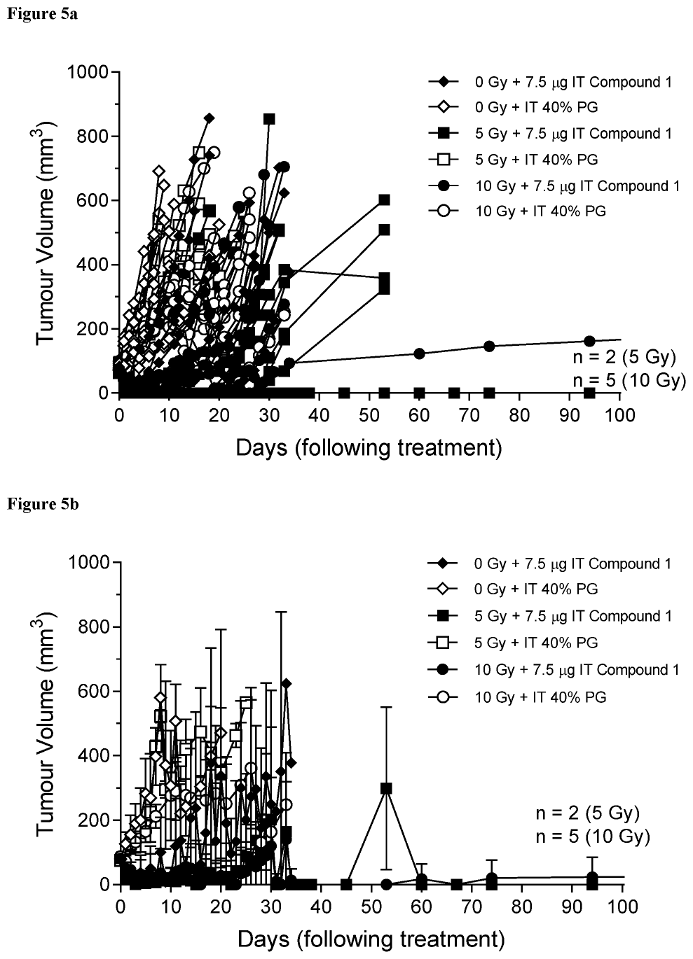

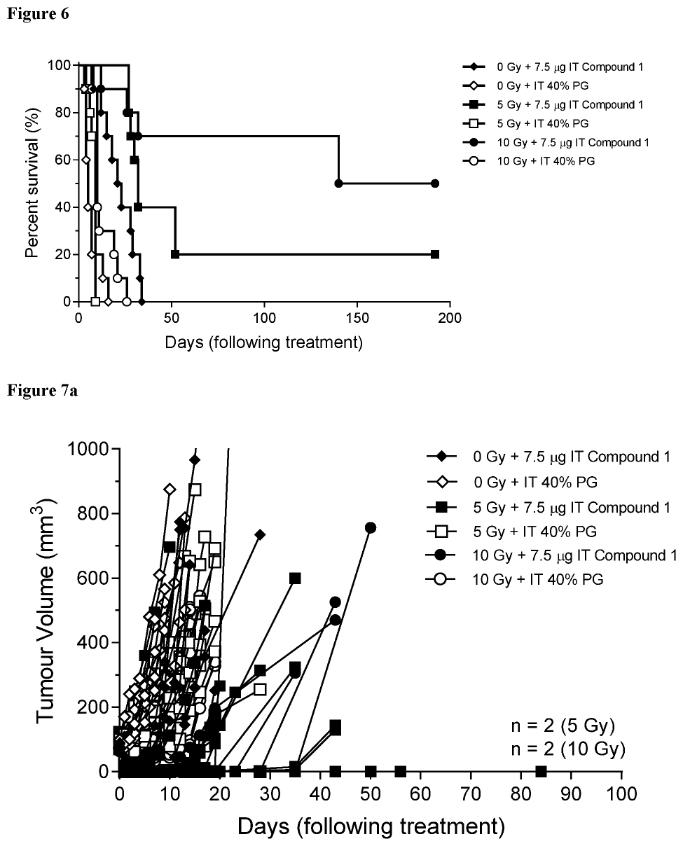

Individual SCC-15 tumour volumes and average tumour volumes for the combination of Compound 1 and irradiation in the BALB/c Foxn1^{mu} mouse model are shown in FIGS. 5a and 5b respectively. Kaplan-Meier survival curves for the xenogeneic model are shown in FIG. 6.

The BALB/c Foxn1^{nu} mouse model is an immunocompromised host that lacks an intact acquired immune system. This model allows assessment of the Compound 1/irradiation combination therapy where the host immune system is less likely to be contributing to any treatment effect.

The individual and average tumour volumes in FIGS. 5a and 5b demonstrate that Compound 1 when combined with irradiation leads to a significant and durable reduction in tumour size compared to other combinations. The combination of 10 Gy and 7.5 μg IT Compound 1 provided the best result and 5 Gy irradiation and 7.5 μg IT Compound 1 delayed tumour growth. The next best results were seen with either 7.5 μg IT Compound 1 or 10 Gy of irradiation, both of which induced delayed tumour response. 10 Gy irradiation induced a delayed tumour response with an average tumour volume showing an initial steady decrease before plateauing and regressing slightly between day 20 and 25. Tumours began to relapse following day 25 and average tumour volume began to increase. There was little difference evident between treatment with 5 Gy irradiation alone and the no treatment control.

The findings in FIGS. 5a and 5b are replicated in the Kaplan-Meier survival curve in FIG. 6. FIG. 6 demonstrates that treatment with combinations of Compound 1 and irradiation gives a significant survival benefit over monotherapy treatments. In this xenogeneic model, the higher dosed Compound 1 and irradiation combination has a 50% survival rate at 200 days while the 5 Gy combination with Compound 1 shows a 20% survival rate. Other treatments show progressive and steady fall in survival over the first 30 days following treatment.

### Syngeneic Model

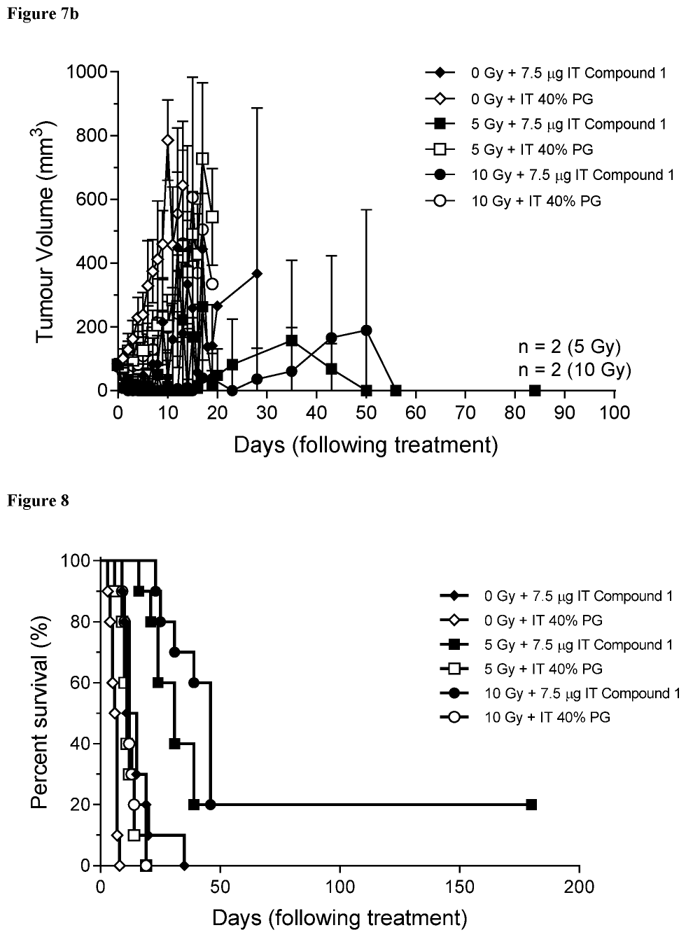

Individual MC38 tumour volumes and average tumour volumes for the combination of Compound 1 and irradiation in the C57BL/6 syngeneic mouse model are shown in FIGS. 7a and 7b respectively. Kaplan-Meier survival curves for the syngeneic mouse model are shown in FIG. 8.

C57BL/6 mouse model inoculated with MC38 tumours is a syngeneic model where the mouse is immunocompetent and the influence of the immune system on treatment may be assessed.

In the syngeneic model, combinations of Compound 1 and irradiation again lead to a considerably improved tumour response as evidenced by the individual and average tumour volumes in FIGS. 7a and 7b. For each of the Compound 1 and irradiation combinations, there were two tumours that remained cured at 200 days following treatment. The remaining tumours all recurred and showed rapid growth at 20 to 50 days following treatment. Despite the early relapses, the results for combination therapy remained far superior to the other single agent treatment regimens.

The Kaplan-Meier survival analysis in FIG. 8 shows that Compound 1 and irradiation treatment is associated with superior survival than other treatment combinations. The two combinations of Compound 1 and irradiation (5 and 10 Gy) have a similar long-term survival with each having a 20% survival at experiment end. Each agent used alone appears to have similar rates of survival.

Irradiation by itself, either 5 Gy or 10 Gy, had a cure rate of 0%. However, the addition of Compound 1 to irradiation resulted in a significantly improved cure rate. The proportion of tumours responding completely to Compound 1 and 5 Gy or 10 Gy irradiation was 80% and 70% respectively for the xenogeneic model and 100% for the syngeneic model. 7.5 μg IT Compound 1 alone resulted in a 70% cure rate for both mouse models. Whilst the cure rate for Compound 1 alone is considerable and close to that of combination treatment, a primary difference between the monotherapy and the combination therapy is the duration of response. Compound 1 with irradiation, either 5 Gy or 10 Gy, improved duration of treatment response at least 4.6× for the xenogeneic model (11 vs 51.5 and 111 days) and 8.7× for the syngeneic model (6.2 vs 64.6 and 65.9 days).

Median survival with each treatment is shown in Table 8.

TABLE 8

| | Median Survival (days) | |
|---|---|---|
| Treatment | Xenogeneic Model | Syngeneic Model |
| 0 Gy + IT 40% PG | 6 | 6.5 |
| 5 Gy + IT 40% PG | 9 | 11 |
| 10 Gy + IT 40% PG | 10 | 12 |
| 0 Gy + IT 7.5 μg Compound | 22 | 13 |
| 1 | | |
| 5 Gy + IT 7.5 μg Compound | 32 | 31 |
| 1 | | |
| 10 Gy + IT 7.5 μg | 166 | 47 |
| Compound 1 | | |

### Example 3: In Vitro Experiments with Compound 1 and Chemoirradiation

The cell lines were investigated in in vitro experiments for assessment of combinations of Compound 1 with cisplatin, 5-fluorouracil and irradiation. The cell lines are given in Table 9 and include one cutaneous cell line and one oropharyngeal mucosal cell line.

TABLE 9

| Cell line | Derivation | Origin | Source |
|---|---|---|---|
| A431 | Human | Epidermoid carcinoma cell line from 85 | ATCC (CRL- |
| | | yr old Caucasian female (1973) | 1555) |
| SCC-15 | Human | Tongue tumour tissue from a 55 yr old | ATCC (CRL- |
| | | Caucasian male (1981) | 1623) |

### Cell Culture

Cultured in Roswell Park Memorial Institute (RPMI-1640; CSL Biosciences, Parkville, Victoria, Australia) media containing 10% (v/v) foetal calf serum (FCS, CSL Biosciences), 100 μg/mL streptomycin with 60 μg/mL penicillin (CSL Biosciences), 1 mM pyruvate, 0.2 mM nicotinamide, and 3 mM 4-(2-hydroxyethyl)piperazine-1-ethanesulfonic acid (HEPES), cells were incubated at 37° C. with 5% carbon dioxide and 95% humidity (Heracell Vios 160i CO₂ incubator; Thermo Scientific, Massachusetts, USA). Twice weekly, cell line confluence was assessed and if approaching 90%, passaged with removal of media before washing with phosphate buffered saline (PBS, 137 mM NaCl, 6.5 mM Na₂HPO₄, 1.5 mM KH₂PO₄, pH 7.4, QIMR Media Services) and treating with 0.25% (v/v) trypsin (Invitrogen, California, USA) and 3.8% (v/v) versine in Dulbecco's salt solution (QIMR Media Services) to allow lifting of the cells. An appropriate volume, a 1:5 to 1:10 ratio, of cells was removed with the residual re-incubated in new RPMI-1640/FCS.

The cell lines were authenticated by short tandem repeat (STR) profiling and mycoplasma testing as performed by the QIMR Analytical Facility.

The number of cells removed during passage was determined using a haemocytometer (Fuchs Rosenthal Dark Lines; Hirschmann Laboratories, Eberstadt, Germany). Cells were seeded at 3000 cells/well in 100 μL of medium in flat bottom 96 well cell culture plates (Corning Incorporated, New York, USA). Cells were incubated for 24 hr to ensure plate adherence prior to treatment.

### Single Agent Exposure

Each cell line was exposed to a range of dosages of Compound 1 (20 μg/mL in 100% ethanol), cisplatin (50 μM stock in 0.9% normal saline, Sigma Aldrich, Missouri, USA), 5-fluorouracil (5-FU, 50 μM stock in dimethylsulfoxide (DMSO, Sigma Aldrich), and irradiation. Compound 1 concentration was confirmed by HPLC prior to use. Dose ranges are provided in Table 10.

TABLE 10

| Agent | Dose Range | |
|---|---|---|
| Compound 1 | 0-500 | μM |
| Cisplatin | 0-200 | μM |
| 5-Fluorouracil | 0-200 | μM |
| Irradiation | 0-8 | Gy |

For each of Compound 1, cisplatin and 5-FU, media from the superior most row of the 96-well plate was aspirated prior to administration of 200 μL of respective drug stock in RPMI-1640 media formulated to the maximal desired concentration. One in two dilutions were then performed down the plate leaving the last row as an untreated control. There was an ethanol control for each cell line which was administered in a similar fashion to the various drug compounds. After either 4, 6 or 24 hours, drug was washed off with PBS and 100 μL of new RPMI-1640 media was administered.

Each cell line was exposed to 0-8 Gy of irradiation with Caesium 137 (Gammacell 40 Exactor Theratronics, Ontario, Canada) with 2 Gy intervals at a rate of 1 Gy/min. The media was replaced immediately after treatment.

Each cell line was treated with each agent in triplicate with data being averaged across the three trials.

### Combination Exposure

The cell lines were exposed to combination of Compound 1 and other agent across a range of dosages as shown in Table 11. The dosages were those that resulted in suboptimal cell death (<IC₅₀) as evidenced by single agent experimentation.

TABLE 11

| Cell line | Compound 1 | Cisplatin | 5-Flurouracil | Irradiation |
|---|---|---|---|---|
| A431 | 0, 10, 50, 100, | 0-10 μM | 0-100 μM | 0-8 Gy |
| | 200 μM | | | |
| SCC-15 | 0, 2, 20, 40, 60 | 0-10 μM | 0-100 μM | 0-8 Gy |
| | μM | | | |

Combination experiments between Compound 1 and cisplatin or 5-FU were conducted using varying administration time-courses. To assess the influence of administration time course on treatment efficacy, each cell line was exposed to Compound 1 24 hours before, concurrently and 24 hours after treatment with either cisplatin or 5-FU.

Cell lines treated with a combination of Compound 1 and irradiation were first treated with the allocated duration of irradiation before immediate treatment with Compound 1. For single agent experiments, wells included no treatment and ethanol treatment controls. Following treatment, cells were incubated for 4 hours prior to washing with PBS and replacement of medium.

### Sulforhodamine B Proliferation Assay

Following incubation of treated cells for 5-7 days, cell survival was determined using a sulforhodamine B (SRB) proliferation assay. This assay quantitatively assesses the degree of protein staining as a surrogate marker for cellular growth. Cells were washed with PBS and subsequently fixed with methylated spirits (Chem Supply, South Australia) for at least five minutes. Following fixation, the methylated spirits was removed and cells again washed gently with water prior to staining with 50 μL/well of 0.4% SRB solution (Sigma Aldrich) in 1% acetic acid for sixty minutes. This solution was tipped off and cells quickly washed twice with 1% acetic acid. The assay was completed with administration of 100 μL/well of 10 mM Tris base (unbuffered, pH >9, Invitrogen). Absorbance was measured on an enzyme-linked immunosorbent assay (ELISA) reader (VERSA max microplate reader, Molecular Devices, California, USA) at 564 nm and a five second agitation. Cell survival was graphed as a percentage of control well absorbance against dose.

### Statistical Analysis

Data was analysed utilising Microsoft Excel (2016, Prism (Version 7.0, Graphpad software 2016), and Combusyn (Version 1.0, Chou & Martin 2005). Means, standard deviations and Chou-Talalay combination indices (CI) (Chou (2010)) were determined and used to detect any significant difference between controls and test combination as well as to assess the relationship between Compound 1 and the chemoradiation.

The Chou-Talalay combination theorem is a statistical method by which the effect of several agents when used in combination can be quantified by producing a number relative to 1. Combination-effect (CI-Fa) plots depict the combination indices for a range of drug concentrations graphically. Data points that fall below the line (the line represents a combination index of 1) suggest a synergistic relationship. A benefit of the CI-Fa plot is that it demonstrates a trend across a range of effect levels. In the setting of this example, the x axis or effect (Fa) correlates directly with cell survival. For example, an effect (Fa) of 0.1 is consistent with a cell survival of 10%. Analysis was performed using the software program Combusyn.

### Results

### Single Agents

Using a 4 hour exposure time, cisplatin alone had an IC₅₀ between 3.125 and 6.25 μM in both cell lines. SCC-15 was closer to the lower end, whereas A431 was slightly more resistant and had an IC₅₀ closer to the upper end.

Using a 4 hour exposure time, Compound 1 alone had an IC₅₀ between 62.5 and 200 μM, with SCC-15 closer to the lower end and A431 being slightly more resistant and closer to the upper end.

Using a 4 hour exposure time, 5-FU alone had an IC₅₀ between 12.5 and 50 μM in both cell lines. A431 being slightly more sensitive and closer to the lower end, whereas SCC-15 was slightly more resistant and had an IC₅₀ closer to the upper end.

Exposure to increasing concentrations of irradiation resulted in a linear decrease in cell survival for both cell lines, both having an IC₅₀ of about 4 Gy.

As for the single agent experimentation, the cell lines were maintained in culture before their eventual transfer to 96-well plates. Treatment for the combination experiments involved first exposing cells to either cisplatin, 5-FU or irradiation before immediate 4 hour treatment with five different concentrations of Compound 1. Cell survival was determined relative to no-treatment controls after 7 days of incubation using SRB assays.

### Combinations

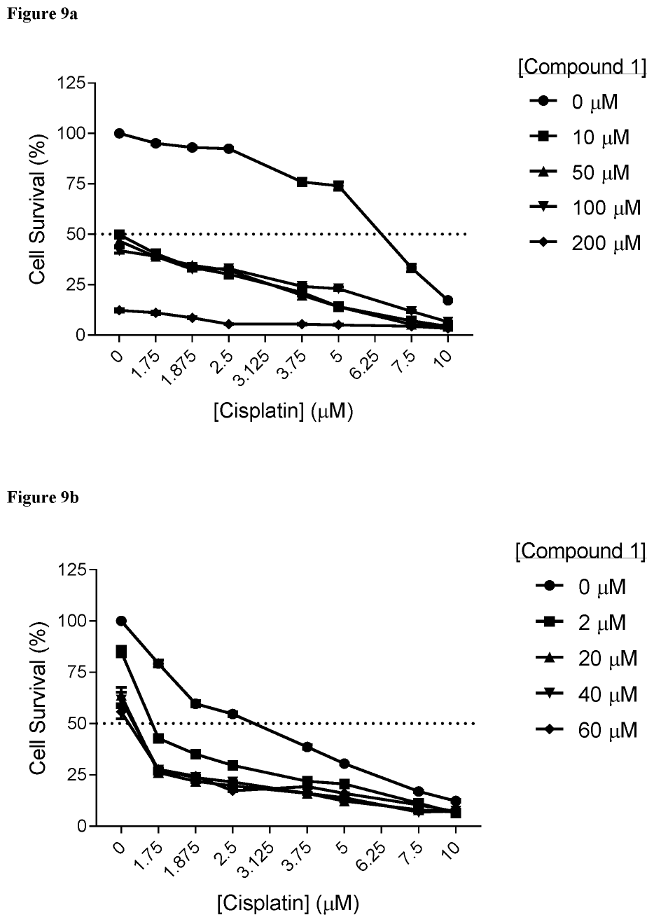

The cell survival for the combination of Compound 1 and cisplatin in A431 cells is shown in FIG. 9a. It is evident that addition of Compound 1 to any concentration of cisplatin results in a significant increase in cell death compared to cisplatin alone. The approximate IC₅₀ for the combination therapy was equivalent to or less than 1.75 μM. There was little difference in efficacy between most of the concentrations of Compound 1, with the exception being 200 μM.

The cell survival for the combination of Compound 1 and cisplatin in SCC-15 cells is shown in FIG. 9b. It is evident that addition of Compound 1 to any concentration of cisplatin results in a significant increase in cell death compared to cisplatin alone. The approximate IC₅₀ for the combination therapy was equivalent to or less than 1.75 μM.

In summary, combination treatment with cisplatin and Compound 1 showed a trend whereby increases in concentration of cisplatin, irrespective of the Compound 1 concentration, resulted in a consequent decrease in cell survival for both cell lines. The addition of Compound 1, even at very low concentrations, resulted in a decreased cell survival as compared to those cells exposed to cisplatin alone. For both cell lines, the IC₅₀ for the combination was reduced compared to that of the single agent alone.

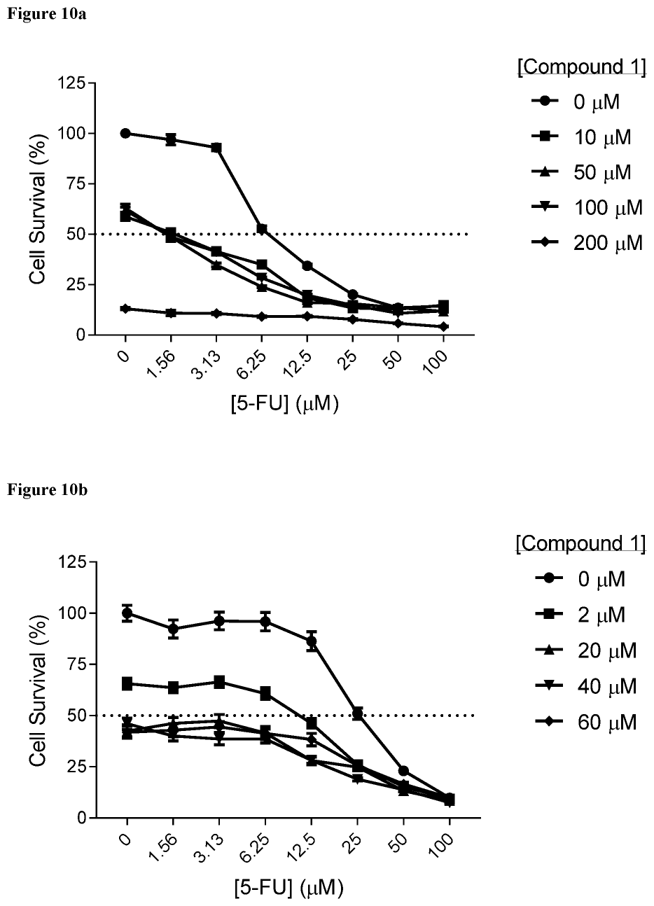

The cell survival for the combination of Compound 1 and 5-FU in A431 cells is shown in FIG. 10a. It is evident that addition of Compound 1 to any concentration of 5-FU results in a significant increase in cell death compared to 5-FU alone. The approximate IC₅₀ for the combination therapy was equivalent to or less than 1.56 μM.

The cell survival for the combination of Compound 1 and 5-FU in SCC-15 cells is shown in FIG. 10b. It is evident that addition of Compound 1 to any concentration of 5-FU results in a significant increase in cell death compared to cisplatin alone. The approximate IC₅₀ for the combination therapy was equivalent to or less than 12.5 μM.

In summary, A431 cells and SCC-15 cells demonstrated a similar response with increases in both 5-FU and Compound 1 resulting in decreased cell survival.

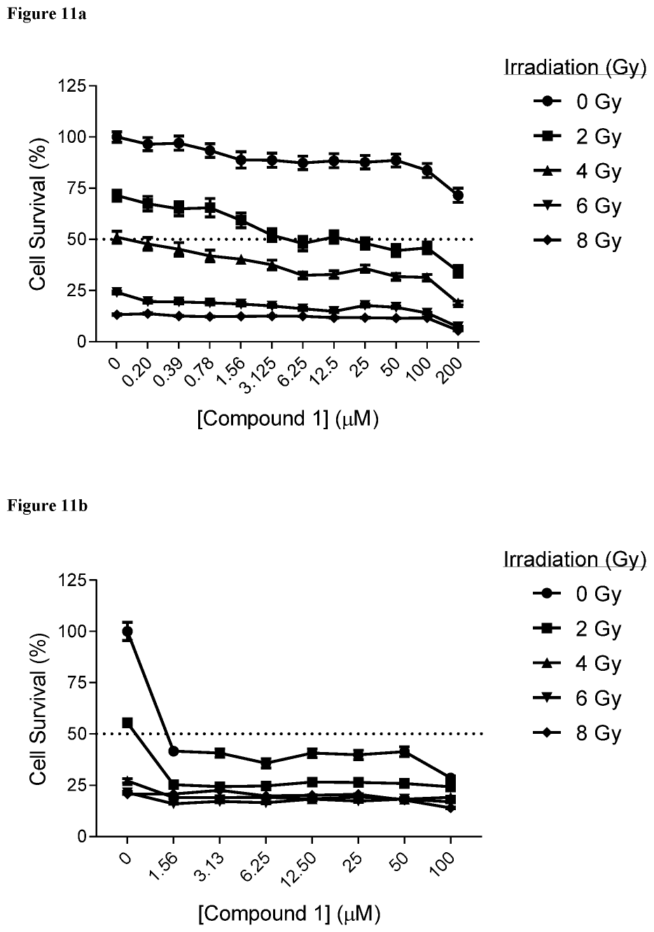

The cell survival for the combination of Compound 1 and irradiation in A431 cells is shown in FIG. 11a. It is evident that addition of Compound 1 to any concentration of irradiation results in a significant increase in cell death compared to Compound 1 alone.

The cell survival for the combination of Compound 1 and irradiation in SCC-15 cells is shown in FIG. 11b. Exposure to Compound 1 and irradiation results in a pronounced fall in cell survival. The combination is superior to that of Compound 1 alone. Increasing irradiation exposure does not greatly improve efficacy.

The IC₅₀ for the combination of Compound 1 and irradiation was 0 to 6.26 μM for A431 cells and 0 to 1.56 μM for SCC-15 cells.

### Order of Administration

For a combination therapy with Compound 1 and cisplatin, there was little difference between administration of Compound 1 24 hours before or 24 hours after cisplatin in A431 cells. However, concurrent therapy improved efficacy significantly over all concentrations of Compound 1.

For a combination therapy with Compound 1 and cisplatin, there was little difference between administration of Compound 1 24 hours before or 24 hours after cisplatin or concurrent administration in SCC-15 cells.

For a combination therapy with Compound 1 and 5-FU in A431 cells, there was some variability in efficacy. Concurrent therapy results in the greatest cell death across the range of concentrations of Compound 1. Pre-treatment with Compound 1 is the next efficacious with cell survival nearing that of concurrent treatment at higher concentrations of 5-FU.

For a combination therapy with Compound 1 and 5-FU, there was little difference between administration of Compound 1 24 hours before or 24 hours after 5-FU or concurrent administration in SCC-15 cells.

### Combination Effect

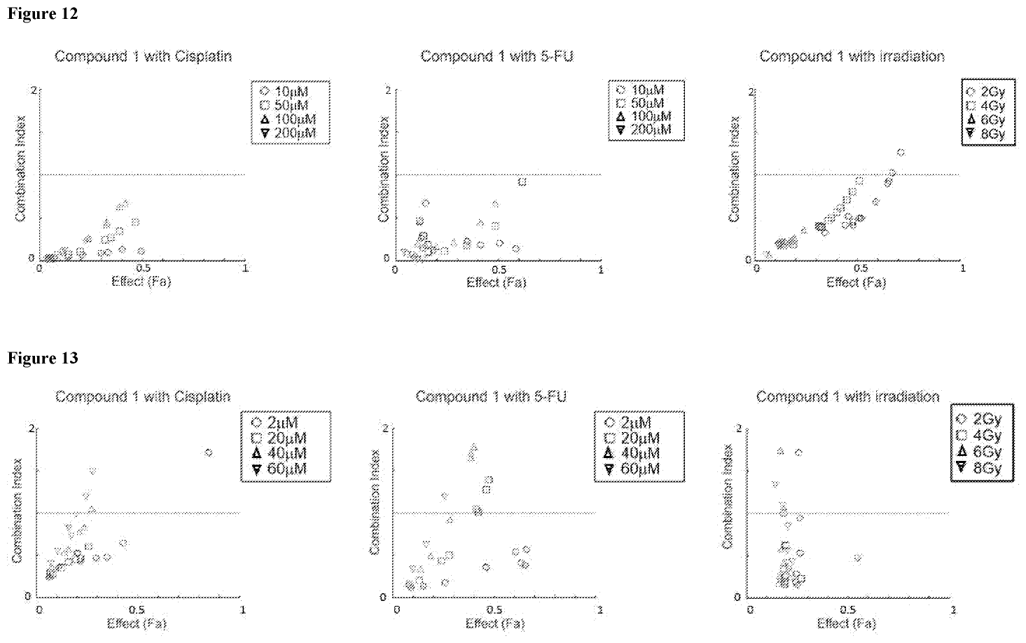

The Combination Effect plot for A431 cells is shown in FIG. 12 and the combination indices for the IC₅₀s for each concentration of Compound 1 is shown in Table 12.

TABLE 12

| Compound 1 | Combination Index at IC₅₀ | | |
|---|---|---|---|
| concentration (μM) | Cisplatin | 5-FU | Irradiation |
| 10 | 0.07 | 0.12 | 0.33 |
| 50 | 0.11 | 0.18 | 0.41 |
| 100 | 0.26 | 0.22 | 0.12 |
| 200 | 0.04 | 0.08 | 0.08 |

Using the Chou-Talalay combination index method, these results suggest that in A431 cells, Compound 1 has a synergistic relationship with cisplatin, 5-FU and irradiation. All data points for the combinations have indices <1.

The Combination Effect plot for SCC-15 cells is shown in FIG. 13 and the combination indices for the IC₅₀s for each concentration of Compound 1 is shown in Table 13.

TABLE 13

| Compound 1 | Combination Index at IC₅₀ | | |
|---|---|---|---|
| concentration (μM) | Cisplatin | 5-FU | Irradiation |
| 2 | 0.67 | 0.18 | 0.15 |
| 20 | 0.61 | 0.44 | 2.1 |
| 40 | 1.05 | 0.5 | 1.75 |
| 60 | 1.49 | 0.63 | 1.34 |

Using the Chou-Talalay combination index method, the results with SCC-15 cells indicate that Compound 1 has a synergistic relationship with cisplatin, 5-FU and irradiation at low concentrations and at all concentrations when combined with 5-FU. However, some combination indices for Compound 1 with irradiation was consistent with antagonism (CI >1).

### Example 4: Assessment of Efficacy of Combined Chemotherapy Treatment with Compound 1 in Mouse Models of Cancer

This example assessed the impact of the chemotherapeutic agent, doxorubicin, in combination with Compound 1 in the treatment on mouse models of cancers, specifically melanoma and lung cancer.

### Description of Methods

### Cell Lines

B16-F10-OVA (B16-F10 mouse melanoma cell line overexpressing chicken ovalbumin) were cultured in RPMI-1640 supplemented with 10% FCS (complete medium) and 3 mM HEPES. LLC (Lewis Lung Carcinoma) cells were cultured in DMEM (Gibco) supplemented with 10% FCS. Both cell lines were incubated at 37° C., 5% CO₂ in a humidified incubator. Routine mycoplasma tests were assessed using Myco-Alert (Lonza), and were always negative.

### Animal Experiments

C57BL/6 mice were purchased from the Animal Resources Centre (ARC), Murdoch, Western Australia. All animal procedures were approved in accordance with NHMRC guidelines (Australian Code for the Care and Use of Animals for Scientific Purposes 8^{th} Edition, 2013; National Health and Medical Research Council of Australia) by the QIMR Berghofer Animal Ethics Committee, approval number A2109-621. All mice were housed in a specific pathogen free (SPF) facility, with 12 hours light/dark cycle and continual access to food and water.

### Doxorubicin Combination Treatment

C57BL/6 mice (6-7 weeks old) were injected subcutaneously (s.c.) on both flanks with B16-F10-OVA mouse melanoma cells (2×10⁵ cells per site in 50 μl) or LLC mouse lung cancer cells (5×10⁵ cells per site in 50 μl). Tumours were allowed to develop to approximately 75-100 mm³. Mice were then treated intra-peritoneally with doxorubicin in saline (3 mg/kg; 60 μg/dose, saline for vehicle). The following day, tumours were injected intratumourally with either vehicle (40% PG/30 mM acetate) or 5 or 10 μg Compound 1 (50 μl of 100 μg/ml [5 μg] or 50 μl of 200 μg/ml [10 μg]) in the 40% PG/30 mM acetate pH 4.30 as previously published (Cullen et al. 2021). Treatment with the chemotherapy agent was repeated after 7 and 14 days. For the B16-F10-OVA model, 5 mice per group (10 tumours) were used in duplicate experiments (total of 10 mice per group, 20 tumours in total from both experiments). For the LLC tumour model, 5 mice per group were used in a single experiment (10 tumours).

Volumes were recorded using digital calipers and expressed as mm³ according to the formula A×b×b×0.5 where A=length and b=measured breadth of the tumour. Mice were also assessed for clinical signs of distress according to an approved clinical score sheet during the experiment. The experiment ceased when an unacceptable clinical score (>6) was reached, or the cumulative tumour burden of the mouse exceeded 1,000 mm³. All mice with ablated tumours from the experimental regimes above will be monitored for an additional 2 months to confirm cure or tumour recurrence/metastasis. Mice were humanely euthanized by CO₂ asphyxiation at the end of the experiment.

### Results

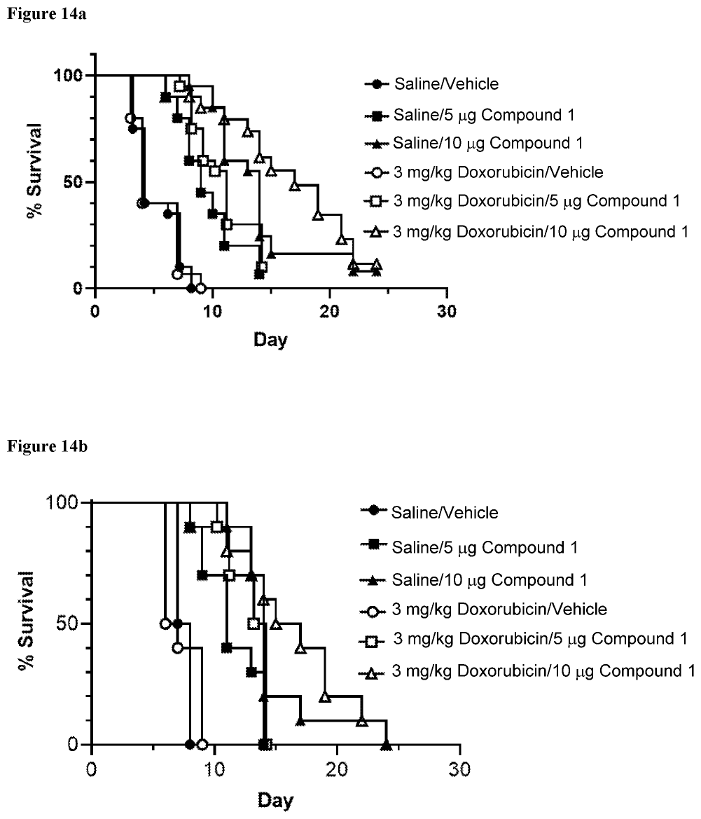

Treatment of B16-F10-OVA (FIGS. 14a and 14b) tumours in C57BL/6 mice with either intraperitoneal doxorubicin or saline in combination with intralesional injection of 40% PG/30 mM acetate had no effect on tumour growth. As expected, treatment with either 5 μg or 10 μg Compound 1 slowed tumour growth and prolonged overall survival compared to vehicle alone (p<0.0001, not shown in FIG. 14b). Further, in the B16-F10-OVA model, doxorubicin treatment in combination with 10 μg Compound 1 had an effect on tumour growth, with extending time until experimental endpoint when compared to doxorubicin alone and to a lesser extent 10 μg Compound 1 combined with saline vehicle. A total of 7/20 tumours were completely ablated in the doxorubicin/10 μg Compound 1 cohort compared to 4/20 tumours in the saline/10 μg Compound 1 group.

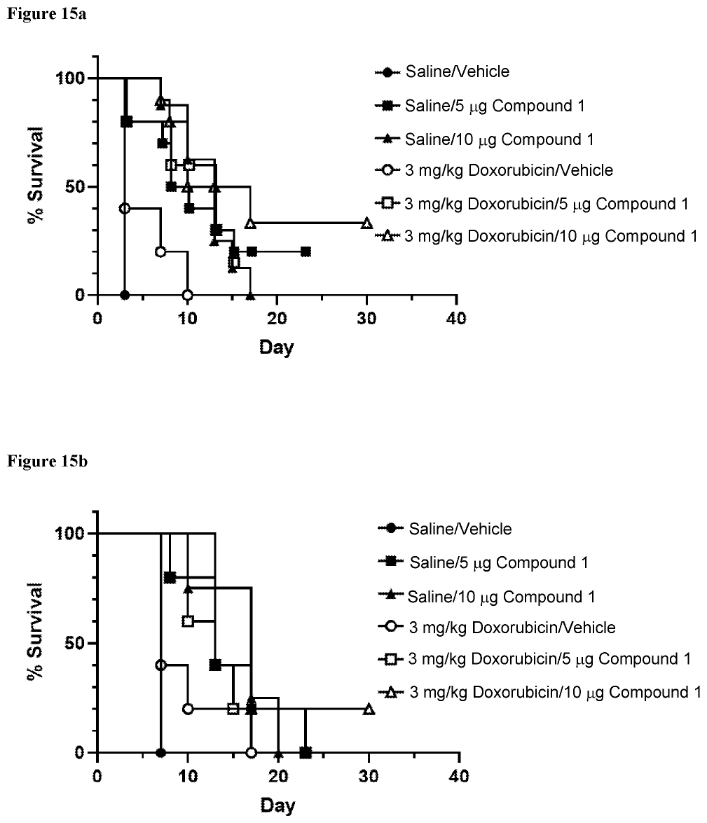

Treatment of LLC (FIGS. 15a and 15b) tumours in C57BL/6 mice with either intraperitoneal doxorubicin or saline in combination with intralesional injection of 40% PG/30 mM acetate had a minor effect on slowing tumour growth and prolonging survival. Again, treatment with either 5 μg or 10 μg Compound 1 slowed tumour growth and prolonged overall survival compared to vehicle alone (p<0.0001, not shown in FIG. 15b). Further, doxorubicin treatment in the LLC model in combination with 10 μg Compound 1 had an effect on tumour growth, with no tumour growth in 3/10 instances. This led to an extension of time until experimental endpoint when compared to doxorubicin alone and to a lesser extent 10 μg Compound 1 combined with saline vehicle.

### Discussion

Combination treatment with doxorubicin in combination with 10 μg Compound 1 had an effect on slowing tumour growth, and prolonging overall survival of C57BL/6 mice with B16-F10-OVA or LLC tumours.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### REFERENCES

Sabrin Husein Albeituni, Chuanlin Ding and Jun Yan. Hampering the Immune Suppressors: Therapeutic Targeting of Myeloid-Derived Suppressor Cells (MDSC) in Cancer. Cancer J. 2013; 19(6): 490-501.
Boyle G M, D'Souza M M, Pierce C J, Adams R A, Cantor A S, Johns J P, Maslovskaya L, Gordon V A, Reddell P W, Parsons P G. Intra-Lesional Injection of the Novel PKC Activator EBC-46 Rapidly Ablates Tumors in Mouse Models. PLoS One 9(10): e108887. https://doi.org/10.1371/journal.pone.0108887.
L Bracci, G Schiavoni, A Sistigu and F Belardelli. Immune-based mechanisms of cytotoxic chemotherapy: implications for the design of novel and rationale-based combined treatments against cancer. Cell Death and Differentiation (2014) 21, 15-25.
Chou T C, Drug combination studies and their synergy quantification using the Chou-Talalay method. 2010, Cancer Research, 70:440-446.
Kahlin Cheung-Ong, Guri Giaever, Corey Nislow DNA-Damaging Agents in Cancer Chemotherapy: Serendipity and Chemical Biology. Chemistry and Biology 20(5), 648-659, 2013.
Shaloam Dasari and Paul Bernard Tchounwou. Cisplatin in cancer therapy: molecular mechanisms of action. Eur J Pharmacol. 2014 Oct. 5; 740: 364-378.
Hall A G, Tilby M J. Mechanisms of action of, and modes of resistance to, alkylating agents used in the treatment of haematological malignancies. Blood Reviews. (1992) 6 (3): 163-73.
Azadeh Mohammadgholi, Azra Rabbani-Chadegani, and Sodabeh Fallah Mechanism of the Interaction of Plant Alkaloid Vincristine with DNA and Chromatin: Spectroscopic Study. DNA Cell Biol. 2013 May; 32(5): 228-235.
Maryam Moudi, Rusea Go, Christina Yong Seok Yien, and Mohd. Nazre. Vinca Alkaloids. Int J Prev Med. 2013 November; 4(11): 1231-1235.
Cullen J K, Boyle G M, Yap P Y, Elmlinger S, Simmons J L, Broit N, Johns J, Ferguson B, Maslovskaya L A, Savchenko A I, Mirzayans P M, Porzelle A, Bernhardt P V, Gordon V A, Reddell P W, Pagani A, Appendino G, Parsons P G, Williams C M. Activation of PKC supports the anticancer activity of tigilanol tiglate and related epoxytiglianes. Sci Rep. 2021 Jan. 8; 11(1):207. doi: 10.1038/s41598-020-80397-9. PMID: 33420238.

1-23. (canceled)

24. A method of treating a tumour comprising administering a combination of an epoxytigliane compound and a second therapeutic agent; wherein the epoxytigliane compound is a compound of formula (I): 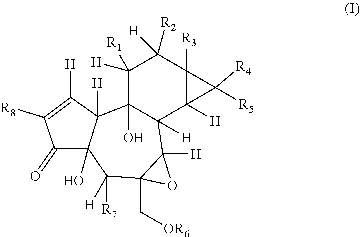
or a geometric isomer or stereoisomer or a pharmaceutically acceptable salt thereof;
wherein
R₁ is hydrogen or C₁₋₆alkyl;
R₂ is —OH or —OR₉;
R₃ is —OH or —OR₉;
R₄ and R₅ are independently selected from hydrogen and C₁₋₆alkyl;
R₆ is hydrogen or —R₁₀;
R₇ is hydroxy or OR₁₀;
R₈ is hydrogen or C₁₋₆alkyl;
R₉ is —C₁₋₂₀alkyl, —C₂₋₂₀alkenyl, —C₂₋₂₀alkynyl, —C(O)C₁₋₂₀alkyl, —C(O)C₂₋₂₀alkenyl, —C(O)C₂₋₂₀alkynyl, —C(O)cycloalkyl, —C(O)C₁₋₁₀alkylcycloalkyl; —C(O)C₂₋₁₀alkenylcycloalkyl, —C(O)C₂₋₁₀alkynylcycloalkyl, —C(O)aryl, —C(O)C₁₋₁₀alkylaryl, —C(O)C₂₋₁₀alkenylaryl, —C(O)C₂₋₁₀alkynylaryl, —C(O)C₁₋₁₀alkylC(O)R₁₁, —C(O)C₂₋₁₀alkenylC(O)R₁₁, —C(O)C₂₋₁₀alkynylC(O)R₁₁, —C(O)C₁₋₁₀alkylCH(OR₁₁)(OR₁₁), —C(O)C₂₋₁₀alkenylCH(OR₁₁)(OR₁₁), —C(O)C₂₋₁₀alkynylCH(OR₁₁)(OR₁₁), —C(O)C₁₋₁₀alkylSR₁₁, —C(O)C₂₋₁₀alkenylSR₁₁, —C(O)C₂₋₁₀alkynylSR₁₁, —C(O)C₁₋₁₀alkylC(O)OR₁₁, —C(O)C₂₋₁₀alkenylC(O)OR₁₁, —C(O)C₂₋₁₀alkynylC(O)OR₁₁, —C(O)C₁₋₁₀alkylC(O)SR₁₁, —C(O)C₂₋₁₀alkenylC(O)SR₁₁, —C(O)C₂₋₁₀alkynylC(O)SR₁₁, 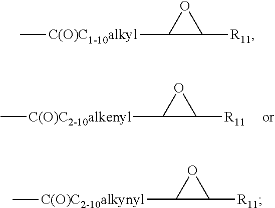
R₁₀is —C₁₋₆alkyl, —C₂₋₆alkenyl, —C₂₋₆alkynyl, —C(O)C₁₋₆alkyl, —C(O)C₂₋₆alkenyl, —C(O)C₂₋₆alkynyl, —C(O)aryl, —C(O)C₁₋₆alkylaryl, —C(O)C₂₋₆alkenylaryl, or —C(O)C₂₋₆alkynylaryl; and
R₁₁ is hydrogen, —C₁₋₁₀alkyl, —C₂₋₁₀alkenyl, —C₂₋₁₀alkynyl, cycloalkyl, or aryl;
wherein each alkyl, alkenyl, alkynyl, cycloalkyl or aryl group is optionally substituted; and
wherein the second therapeutic agent is selected from:
i) a chemotherapeutic agent that damages DNA;
ii) a chemotherapeutic agent that inhibits tumour-associated host-derived cells that support the growth and/or invasion of tumour cells; and
iii) irradiation.

25. The method according to claim 24, wherein the epoxytigliane compound is administered locally to the tumour.

26. The method according to claim 25, wherein the local administration is by intratumoural injection.

27. The method according to claim 24, wherein the second therapeutic agent is a chemotherapeutic agent that damages DNA.

28. The method according to claim 27, wherein the chemotherapeutic agent is selected from a platinum chemotherapeutic agent, a DNA intercalating agent, a DNA alkylating agent, an antimetabolite, or a compound that binds to GC rich DNA.

29. The method according to claim 24, wherein the second therapeutic agent is a chemotherapeutic agent that inhibits tumour-associated host-derived cells that support the growth and/or invasion of tumour cells.

30. The method according to claim 29, wherein the chemotherapeutic agent is a vinca alkaloid, an antimetabolite, a phosphodiesterase 5 inhibitor, amiloride, a COX-2 inhibitor or a PEG-2 inhibitor.

31. The method according to claim 24, wherein the second therapeutic agent is irradiation.

32. The method according to claim 24, wherein in the epoxytigliane compound of formula (I), one or more of the following applies:
i) R₁ is —CH₃;
ii) R₂ and R₃ are independently selected from —OC(O)C₁₋₂₀alkyl, —OC(O)C₂₋₂₀alkenyl, —OC(O)C₂₋₂₀alkynyl, —OC(O)cycloalkyl, —OC(O)C₁₋₁₀alkylcycloalkyl, —OC(O)C₂₋₁₀alkenylcycloalkyl, —OC(O)C₂₋₁₀alkynylcycloalkyl, —OC(O)aryl, —OC(O)C₁₋₁₀alkylaryl, —OC(O)C₂₋₁₀alkenylaryl, —OC(O)C₂₋₁₀alkynylaryl, —OC(O)C₁₋₁₀alkylC(O)R₁₁, —OC(O)C₂₋₁₀alkenylC(O)R₁₁, —OC(O)C₂₋₁₀alkynylC(O)R₁₁, —OC(O)C₁₋₁₀alkylCH(OR₁₁)(OR₁₁), —OC(O)C₂₋₁₀alkenylCH(OR₁₁)(OR₁₁), —OC(O)C₂₋₁₀alkynylCH(OR₁₁)(OR₁₁), —OC(O)C₁₋₁₀alkylSR₁₁, —OC(O)C₂₋₁₀alkenylSR₁₁, —OC(O)C₂₋₁₀alkynylSR₁₁, —OC(O)C₁₋₁₀alkylC(O)OR₁₁, —OC(O)C₂₋₁₀alkenylC(O)OR₁₁, —OC(O)C₂₋₁₀alkynylC(O)OR₁₁, —OC(O)C₁₋₁₀alkylC(O)SR₁₁, —OC(O)C₂₋₁₀alkenylC(O)SR₁₁, and —OC(O)C₂₋₁₀alkynylC(O)SR₁₁;
iii) R₄ and R₅ are each independently selected from H and —CH₃;
iv) R₆ is hydrogen, —C(O)C₁₋₆alkyl, —C(O)C₂₋₆alkenyl, —C(O)C₂₋₆alkynyl, or —C(O)aryl;
v) R₇ is hydroxyl, —OC(O)C₁₋₆alkyl, —OC(O)C₂₋₆alkenyl, or —OC(O)C₂₋₆alkynyl; and
vi) R₈ is H or —CH₃.

33. The method according to claim 32, wherein R₂ and R₃ are independently selected from —OC(O)C₁₋₂₀alkyl, —OC(O)C₂₋₂₀alkenyl, —OC(O)C₂₋₂₀alkynyl, —OC(O)cycloalkyl, —OC(O)C₁₋₁₀alkylcycloalkyl; —OC(O)C₂₋₁₀alkenylcycloalkyl, —OC(O)C₂₋₁₀alkynylcycloalkyl, and —OC(O)aryl.

34. The method according to claim 32, wherein R₂ and R₃ are independently selected from —OC(O)C₁₋₂₀alkyl, —OC(O)C₂₋₂₀alkenyl, and —OC(O)C₂₋₂₀alkynyl.

35. The method according to claim 24, wherein the compound of formula (I) is a compound of formula (II): 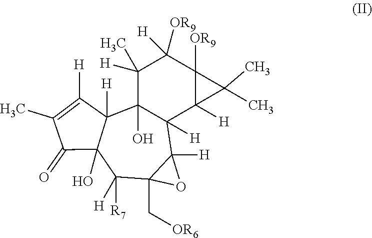
or a geometric isomer or stereoisomer or a pharmaceutically acceptable salt thereof,
wherein R₆, R₇ and R₉ are as defined in claim 1.

36. The method according to claim 24, wherein the epoxytigliane compound of formula (I) is selected from the following compounds:
12-tigloyl-13-(2-methylbutanoyl)-6,7-epoxy-4,5,9,12,13,20-hexahydroxy-1-tiglien-3-one;
12,13-di-(2-methylbutanoyl)-6,7-epoxy-4,5,9,12,13,20-hexahydroxy-1-tiglien-3-one;
12-hexanoyl-13-(2-methylbutanoyl)-6,7-epoxy-4,5,9,12,13,20-hexahydroxy-1-tiglien-3-one;
12,13-dihexanoyl-6,7-epoxy-4,5,9,12,13,20-hexahydroxy-1-tiglien-3-one;
12-myristoyl-13-(2-methylbutanoyl)-6,7-epoxy-4,5,9,12,13,20-hexahydroxy-1-tiglien-3-one;
12-tigloyl-13-(2-methylbutanoyl)-6,7-epoxy-4,5,9,12,13-pentahydroxy-20-acetyloxy-1-tiglien-3-one;
12-myristoyl-13-acetyloxy-6,7-epoxy-4,5,9,12,13,20-hexahydroxy-1-tiglien-3-one;
12-propanoyl-13-(2-methylbutanoyl)-6,7-epoxy-4,5,9,12,13,20-hexahydroxy-1-tiglien-3-one;
12,13-ditigloyl-6,7-epoxy-4,5,9,12,13,20-hexahydroxy-1-tiglien-3-one; and
12-(2-methylbutanoyl)-13-tigloyl-6,7-epoxy-4,5,9,12,13,20-hexahydroxy-1-tiglien-3-one;
or a pharmaceutically acceptable salt thereof.

37. The method according to claim 24, wherein the epoxytigliane compound and the second therapeutic agent are administered separately and simultaneously.

38. The method according to claim 24, wherein the tumour is a cutaneous or subcutaneous tumour.

39. The method according to claim 24, wherein the tumour is a squamous cell carcinoma, an adenocarcinoma, or a sarcoma.

40. The method according to claim 24, wherein the tumour is a head or neck cancer.

41. The method according to claim 39, wherein the tumour is a cutaneous or mucosal squamous cell cancer.

42. The method according to claim 24, wherein the epoxytigliane compound is (4S,5S,6R,7S,8R,9R,10S,11R,12R,13S,14R)-12-(2E)-2-methyl-2-enoatyl-13((2S)-2-methylbutyroyl)-6,7-epoxy-4,5,9,12,13,20-hexahydroxy-1-tigliaen-3-one.

43. The method according to claim 42, wherein the second therapeutic agent is selected from cisplatin, 5-fluorouracil, doxorubicin, and irradiation.
